(19) United States
(12) Patent Application Publication
(10) Pub. No.: US 2017/0037234 A1
(43) Pub. Date: Feb. 9, 2017

(54) POLYMER NANOPARTICLES

(71) Applicant: THE TRUSTEES OF PRINCETON UNIVERSITY, Princeton, NJ (US)

(72) Inventors: Robert K. PRUD'HOMME, Lawrenceville, NJ (US); Rodney D. PRIESTLEY, Princeton, NJ (US); Rui LIU, Princeton, NJ (US); Chris SOSA, Princeton, NJ (US)

(73) Assignee: THE TRUSTEES OF PRINCETON UNIVERSITY, Princeton, NJ (US)

(21) Appl. No.: 15/121,715

(22) PCT Filed: Feb. 25, 2015
(86) PCT No.: PCT/US15/17590

§ 371 Date: Aug. 25, 2016

Related U.S. Application Data
(60) Provisional application No. 61/944,784, filed on Feb. 26, 2014.
(60) Provisional application No. 62/042,515, filed on Aug. 27, 2014.

(51) Int. Cl. C08L 25/06; A61K 47/32; B01J 31/26; C08L 47/00; B01J 31/06; B01J 31/28; A61K 9/16; A01N 25/10

(52) U.S. Cl. CPC C08L 25/06; A61K 9/16; A61K 47/32; A01N 25/10; C08L 47/00; B01J 31/06; B01J 31/28; B01J 31/26; B01J 2231/641

(57) ABSTRACT

Polymer nanoparticles, including Janus nanoparticles, and methods of making them are described.

This invention was made with government support under Grant No. DMR-0819860 awarded by the National Science Foundation. The government has certain rights in the invention.

### FIELD OF THE INVENTION

The present invention relates to polymer nanoparticles and processes of making them.

### BACKGROUND OF THE INVENTION

In Pierre Gilles de Gennes's 1991 Nobel Laureate speech titled “Soft Matter” he introduced the concept of Janus particles, which are anisotropically structured particles containing two distinct regions of material or functionality. Their development can be considered in the context of the scientific and technological development of other chemically anisotopically structured materials, such as surfactants and block copolymers. The ability to synthesize surfactants at scale and in cost effective ways has led to the current surfactant market. The ability to synthesize block copolymers at scale and in cost effective ways has led to the current market for thermoplastic elastomers based on block copolymers.

Janus colloids can be assembled from a broad variety of building blocks ranging from metals to polymers (Yoon, J. et al., Amphiphilic colloidal surfactants based on electrohydrodynamic co-jetting, ACS Appl. Mater. Interfaces, 2013, 5, 11281-7; Glaser, N. et al., Janus particles at liquid-liquid interfaces, Langmuir 2006, 22, 5227-9). The breadth of material properties exhibited by polymers as well as their ability to phase separate can be useful for the generation of Janus colloids.

Colloids possessing patterned or structured surface domains of differing chemical composition can serve as nanoscale building blocks for the design of materials with molecular scale features (Walther, A. et al., Janus particles. Soft Matter 2008, 4, 663-668; Walther, A. et al. Janus Particles: Synthesis, Self-Assembly, Physical Properties, and Applications, Chem. Rev. 2013, 113, 5194-5261; Samuel, A. Z. et al., Self-Adapting Amphiphilic Hyperbranched Polymers, Macromolecules 2012, 45, 2348-2358). The functionality of such particles can depend on the spatial topology and molecular properties of surface domains (Walther, A. et al., Janus particles, Soft Matter 2008, 4, 663-668; Walther, A. et al., Janus Particles: Synthesis, Self-Assembly, Physical Properties, and Applications, Chem. Rev. 2013, 113, 5194-5261).

Janus nanocolloids can assemble into higher-order superstructures when induced to by various environmental stimuli (Walther, A. et al., Janus particles, Soft Matter 2008, 4, 663-668; Chen, Q. et al., Directed self-assembly of a colloidal kagome lattice, Nature 2011, 469, 381-384). They can, for example, organize under magnetic (Smoukov, S. K. et al., Reconfigurable responsive structures assembled from magnetic Janus particles, Soft Matter 2009, 5, 1285-1292) or electric fields (Gangwal, S. et al., Dielectrophoretic Assembly of Metallodielectric Janus Particles in AC Electric Fields, Langmuir 2008, 24, 13312-13320) to form patterned chains on solid substrates, undergo complex translational (Gangwal, S. et al., Induced-Charge Electrophoresis of Metallodielectric Particles, Phys. Rev. Lett. 2008, 100, 058302) and rotational (Gangwal, S. et al Induced-Charge Electrophoresis of Metallodielectric Particles, Phys. Rev. Lett. 2008, 100, 058302) motion in alternating fields (Squires, T. M. et al., Breaking symmetries in induced-charge electro-osmosis and electrophoresis, J. Fluid Mech. 2006, 560, 65-101), migrate to the interface between two immiscible fluids in order to decrease the surface tension of macroscopic emulsions (Yoon, J. et al., Amphiphilic colloidal surfactants based on electrohydrodynamic co-jetting, ACS Appl. Mater. Interfaces 2013, 5, 11281-7), and uniquely interact with cellular interfaces in order to facilitate the absorption of imaging or therapeutic agents (Gao, Y. et al., How half-coated janus particles enter cells, J. Am. Chem. Soc. 2013, 135, 19091-4).

The interest in multi-faced nanocolloid applications has outstripped the ability to produce commercial-scale materials, hindering the development of new technologies (Samuel, A. Z. et al., Self-Adapting Amphiphilic Hyperbranched Polymers, Macromolecules 2012, 45, 2348-2358; Jang, S. G. et al., Striped, ellipsoidal particles by controlled assembly of diblock copolymers, J. Am. Chem. Soc. 2013, 135, 6649-57; Erhardt, R. et al., Amphiphilic Janus micelles with polystyrene and poly(methacrylic acid) hemispheres, J. Am. Chem. Soc. 2003, 125, 3260-3267; Roh, K. et al., Biphasic Janus particles with nanoscale anisotropy, Nat. Mater. 2005, 4, 759-763; Yamashita, N. et al., Preparation of hemispherical particles by cleavage of micrometer-sized, spherical poly(methyl methacrylate)/polystyrene composite particle with Janus structure: effect of molecular weight, Colloid Polym. Sci. 2013, 292, 733-738).

The scalability of processes for forming and comprehensive control over particle morphology of Janus particles is a challenge (Chang, E. P. et al., Membrane Emulsification and Solvent Pervaporation Processes for the Continuous Synthesis of Functional Magnetic and Janus Nanobeads, Langmuir 2012, 28, 9748-9758; Wang, Y. et al., Colloids with valence and specific directional bonding, Nature 2012, 491, 51-5; Walther, A. et al., Janus discs, J. Am. Chem. Soc., 2007, 129, 6187-98).

Metal nanoparticles (Burda, C. et al., Chem. Rev. 2005, 105, 1025) are typically unstable and tend to sinter into larger species. A suitable carrier is needed to prevent the aggregation of metal nanoparticles (Astruc, D. et al., Angew. Chem. Int. Ed. 2005, 44, 7852.; Na, H. B. et al., Adv. Mater. 2009, 21, 2133). Polymeric matrices (Scott, R. W. et al., J. Am. Chem. Soc. 2004, 126, 15583; Anderson, R. M. et al., ACS Nano 2013, 7, 9345.; Peng, X. et al., Chem. Soc. Rev. 2008, 37, 1619), latex particles (Sun, Q. et al., Langmuir 2005, 21, 5812; Mohammed, H. S. et al., Macromol. Rapid Commun. 2006, 27, 1774; Wong, J. E. et al., J. Colloid Interface Sci. 2008, 324, 47; Ganesan, V. et al. Soft Matter 2010, 6, 4010; Liu, R. et al., ACS Appl. Mater. Interfaces 2013, 5, 9167) have been studied for the immobilization of metal nanoparticles. (Mel, Y. et al, Chem. Mater. 2007, 19, 1062; Schrinner, M. et al., Adv. Mater, 2008, 20, 1928; Lu, Y. et al., Macromol Rapid Comm. 2009, 30, 806.; Wunder, S. et al., J. Phys. Chem. C 2010, 114, 8814; Shenhar, R. et al., Adv. Mater. 2005, 17, 657; Grzelczak, M. et al., ACS Nano 2010, 4, 3591). Nanoparticles, such as magnetic nanoparticles (Krack, M. et al., J. Am. Chem. Soc. 2008, 130, 7315) and quantum dots (Diaz A. et al., Am. Chem. Soc. 2013, 135, 3208) can be encapsulated in various polymer assemblies to form multifunctional materials (Mai, Y. et al., J. Am. Chem. Soc. 2010, 132, 10078.; Jang, S. G. et al., J. Am. Chem. Soc. 135, 6649; Bae, J. et al., Adv. Mater., 2012, 24, 2735; Chen, H. et al., Chem. Phys. Chem. 2008, 9, 388; Li, W. K. et al., Angew. Chem., Int. Ed. 2011, 50, 5865; Kang, Y. et al., Angew. Chem., Int. Ed. 2005, 44, 409; Kim, B. S. et al., Nano Lett. 2005, 5, 1987; Hickey, R. J. et al., J. Am. Chem. Soc. 2011, 133, 1517; Luo, Q. et al., ACS Macro Lett. 2013, 2, 107).

### SUMMARY

Methods according to the invention use Flash NanoPreeipitation (FNP) to produce polymer Janus particles using processes that confine the volume for phase separation. In comparison to existing processes, FNP is a single-step, low energy, continuous, and rapid process that can be used to create polymer:polymer and polymer:inorganic nanoparticles.

For example, polystyrene-block-poly(vinylpyridine) (PS-b-PVP) in tetrahydrofuran (THF), aqueous metal ion salts, and reducing agent solutions (for example, of NaBH₄) can be employed as polymer stream, non-solvent stream, and collection solution, respectively to obtain uniform metal nanoparticles grown on polymer nanospheres through a one-step FNP process. The particle size and metal nanoparticle loading amount can be tuned by changing the preparation parameters (e.g., feeding amount, feeding speed, concentration of polymer in the feed, mixing rate).

Flash Nano Precipitation (FNP) achieves rapid solvent displacement by means of high intensity mixing geometries. Amphiphilic block copolymer chains dissolved in a solvent are mixed with a non-solvent, e.g., typically water, to precipitate and assemble as nanoparticles. For example, metal (e.g. gold (Au) or platinum (Pt)) nanoparticles-nanosphere polymer composites can be generated through FNP. Thus, uniform metal-nanosphere polymer composites with 2-3 nm metal colloids (nanocrystals) decorating the PVP corona on the nanosphere (i.e., PVP polymer constituents on the surface of the nanosphere and PVP polymers and portions of polymers radiating out from surface into the solution) can be obtained with overall particle size and metal nanoparticle arrangement tunable by varying the process parameters. In an exemplary use, the obtained composites show high catalytic ability and stability in the reduction of 4-nitrophenol.

A method according to the invention of forming a multi-faced polymer nanoparticle includes dissolving a first polymer at a first concentration and a second polymer at a second concentration in a solvent to form a polymer solution, selecting a nonsolvent, selecting a mean nanoparticle diameter, selecting the first concentration and second concentration to achieve the selected mean nanoparticle diameter, and continuously mixing the polymer solution with the nonsolvent to flash precipitate the multi-faced polymer nanoparticle in a mixture of the solvent and the nonsolvent. The first polymer can be different from the second polymer. The multi-faced polymer nanoparticle can include a first region, comprising the first polymer at a greater weight fraction than the second polymer, and a second region, comprising the second polymer at a greater weight fraction than the first polymer, with the first region in contact with the second region. In an embodiment, neither the polymer solution nor the nonsolvent include a stabilizer. In a method, the mixing of the polymer solution with the nonsolvent includes mixing with a collection solution. The collection solution can include a stabilizer. The stabilizer can be an amphiphilic surfactant molecule. For example, the stabilizer can be a sulfonated alkyl surfactant, sodium dodecyl sulfate, an ethoxylated sulfonate surfactant, a cationic surfactant, an amine oxide surfactant, a zwitterionic surfactant, an amphoteric surfactant, ethylene oxide surfactant based on an alkyl ether, ethylene oxide surfactant based on a nonylphenol, a surfactant based on sorbitan oleate, glucose-based surfactant, polymeric surfactant, polyethylene oxide-co-polybutylene oxide surfactant, polyvinyl caprolactam based stabilizer, polycaprolactone based stabilizer, polyvinyl alcohol based stabilizer, polyethylene oxide based stabilizer, natural products polymeric stabilizer based on substituted cellulose, hydroxypropyl cellulose, a natural products polymeric stabilizer based on a hydrophobically modified starch, lipid, lecithin, and/or combinations. In an embodiment, the mean particle diameter is in range of from about 30 nm to about 2000 nm, or is in a range of from about 50 nm to about 800 nm. In an embodiment, at least 90% of the nanoparticles formed have a diameter less than 800 nm and at most 10% of the nanoparticles formed have a diameter Less than 50 nm. In an embodiment the first region and the second region together include at least 90% of the total volume. For example, the first region and the second region together can include from about 50%, 70%, 80%, 90%, or 95% to about 70%, 80%, 90%, 95%, or 100% of the total volume.

In a method, the first polymer is polystyrene (PS), polyisoprene (PI), polybutadiene (PB), poly(lactic acid) (PLA), poly(vinylpyridine) (PVP), polyvinylcyclohexane, poly(methyl methacrylate), polycaprolactone, polyamide, polysulfone, epoxy, epoxy resin, silicone rubber, silicone polymer, and/or polyimide. The second polymer can be polystyrene (PS), polyisoprene (PI), polybutadiene (PB), poly(lactic acid) (PLA), poly(vinylpyridine) (PVP), polyvinylcyclohexane (PVCH), poly(methyl methacrylate), polycaprolactone, polyamide, polysulfone, epoxy, epoxy resin, silicone rubber, silicone polymer, and/or polyimide. The first concentration can be in the range of from about 0.01 to about 30 mg/mL. The second concentration can be in the range of from about 0.01 to about 30 mg/mL. The solvent can be selected from tetrahydrofiiran (THF), methyl acetate, ethyl acetate, acetone, methyl ethyl ketone (MEK), dioxane, dimethylformamide (DMF), acetonitrile, methyl pyrrolidone, and dimethyl sulfoxide (DMSO) and/or combinations. The nonsolvent can be selected from the group consisting of water, methanol, ethanol, acetic acid, and/or combinations. In an embodiment, the first polymer is polystyrene (PS), the second polymer is polyisoprene (PI), the solvent is THF, and the nonsolvent is water. In an embodiment, the first polymer is poly(methacrylic acid), the solvent is water, and the nonsolvent is acetone.

In a method, an amphiphilic block copolymer is dissolved in the solvent. The amphiphilic block polymer can include a hydrophobic homopolymer covalently bonded to a hydrophilic homopolymer, with the hydrophobic homopolymer having the same chemical structure as the first polymer. A second amphiphilic block copolymer can be dissolved in the solvent, and the second amphiphilic block polymer can include a second hydrophobic homopolymer covalently bonded to a hydrophilic homopolymer, with the second hydrophobic homopolymer having the same chemical structure as the second polymer.

In a method, the multi-faced polymer nanoparticle is separated from the mixture. For example, the multi-faced polymer nanoparticle can be separated from the mixture by centrifugation, ultrafiltration, and/or spray drying.

In a method, the multi-faced nanoparticle is infused with a medical agent. The medical agent can be a pharmaceutical, an imaging agent, a contrast imaging agent, and/or a radioactive tracer. Alternatively, the multi-faced nanoparticle can be infused with a pesticide or an herbicide.

The first polymer can be a homopolymer or a near homopolymer, the near homopolymer can include a first comonomer and a second comonomer, the first comonomer can be at least 95 wt % of the near homopolymer, and the second comonomer can be at most 5 wt % of the near homopolymer. The mixing of the polymer solution with the nonsolvent can include mixing with a collection solution comprising an anionic surfactant.

An embodiment according to the invention is a group of multi-faced polymer nanoparticles. Each multi-faced polymer nanoparticle can include a first polymer, a second polymer, a first region that includes the first polymer at a greater weight fraction than the second polymer, and a second region that includes the second polymer at a greater weight fraction than the first polymer. The first region can be in contact with the second region. At least 80% of the particles in the group can have a diameter in the range of from about 50 nm to about 800 nm. The first polymer can be a biocompatible polymer.

In a method according to the invention the group of multi-faced polymer nanoparticles is used to strengthen adhesion between a first polymer structure and a second polymer structure at an interface between the first polymer structure and the second polymer structure. The group of multi-faced polymer nanoparticles can be used as an emulsion stabilizer. The group of multi-faced polymer nanoparticles can be used as a foam stabilizer. The group of multi-faced polymer nanoparticles can be used as a foam stabilizer. The group of multi-faced polymer nanoparticles can be used as a solid-liquid interfacial tension modifier.

An embodiment according to the invention is a three-faced polymer nanoparticle that includes a first polymer, a second polymer, and a third polymer, a first region that includes the first polymer at a greater molar fraction than a molar fraction of the second polymer and third polymer, a second region that includes the second polymer at a greater molar fraction than a molar fraction of the first polymer and third polymer, and a third region that includes the third polymer at a greater molar fraction than a molar fraction of the first polymer and second polymer. The first region can be in contact with the second region, and the second region can be in contact with the third region. Each of the first polymer, second polymer, and third polymer can be different from the others. The first region, second region, and third region together can comprise at least 90% of the total volume of the three-faced polymer nanoparticle. The first polymer can be polyvinylcyclohexane (PVCH), the second polymer can be polybutadiene (PB), and the third polymer can be polystyrene (PS).

A method according to the invention is forming a metal-polymer composite nanoparticle by dissolving a polymer in a first solvent at a first concentration to form a polymer solution, dissolving a metal salt in a second solvent at a second concentration to form a metal salt solution, and mixing the polymer solution with the metal salt solution to form the metal-polymer composite nanoparticle with a surface. The metal can be concentrated at the surface. The second solvent can be a nonsolvent for the polymer. The polymer can be a block copolymer, for example, polystyrene-block-poly(vinylpyridine) (PS-b-PVP). For example, the metal can be gold (Au), platinum (Pt), silver (Ag), palladium (Pd), copper (Cu), cobalt (Co), and/or iron (Fe). The mixing of the polymer solution with the metal salt solution can include mixing with a collection solution. The collection solution can include a reducing agent. For example, the reducing agent can be selected from lithium aluminum hydride (LiAlH₄), compounds containing the Sn²⁺ ion, such as tin(II)chloride (SnCl₂), compounds containing the Fe²⁺ ion, such as iron (II) sulfate (FeSO₄), oxalic acid, formic acid, ascorbic acid, sulfite compounds, phosphites, hydrophosphites, phosphorous acid, dithiothreitol (DTT), tris(2-carboxyethyl)phosphine HCl (TCEP), and/or carbon. For example, the reducing agent can be sodium borohydride (NaBH₄). The collection solution can include a stabilizer, for example, sodium dodecyl sulfate (SDS).

In an embodiment according to the invention, a metal-polymer composite nanoparticle includes a core and a shell that surrounds the core. The core can include a polymer, and the shell can include the polymer and a metal. The polymer can be a block copolymer. In a method according to the invention, the metal-polymer composite nanoparticle is used to catalyze a chemical reaction. For example, the chemical reaction catalyzed can be between two immiscible phase liquids.

In an embodiment according to the invention, a multi-faced polymer nanoparticle includes a first polymer and a second polymer, a first region that includes the first polymer at a greater molar fraction than the second polymer, and a second region that includes the second polymer at a greater molar fraction than the first polymer. The first region can be in contact with the second region. The first polymer can be a homopolymer or a near homopolymer, and the near homopolymer can include a first comonomer and a second comonomer. The first comonomer can be at least 95 wt % of the near homopolymer, and the second comonomer can be at most 5 wt % of the near homopolymer.

### BRIEF DESCRIPTION OF THE FIGURES

FIG. 1 shows a schematic of the Flash NanoPrecipitation (FNP) method.

FIGS. 2A and 2B show tunneling electron microscopy (TEM) images of the nanoparticles formed as the overall feed concentration and ratio of polystyrene (PS) and polyisoprene (PI) polymers is varied. FIG. 2A represents nanoparticles formed with lower molecular weight PS and PI polymers, and FIG. 2B represents nanoparticles formed with higher molecular weight PS and PI polymers. FIG. 2C indicates the morphology (Janus or multi-faced) of the nanoparticles formed with respect to the PS/PI ratio and the dimensionless particle diameter along with a comparison to scaling theory.

FIG. 3 shows the Di10, Di50, and Di90 particle diameter values and the Span, indicating particle size distribution, for particles formed as a function of overall feed concentration with a 50:50 mass ratio of polystyrene (PS) and polyisoprene (PI) polymers in the feed.

FIG. 4 shows a tunneling electron microscopy (TEM) image of a tri-lobal Cerberus particle that includes polystyrene, polybutadiene, and polyvinylcyclohexane domains.

FIGS. 5A and 5B show a tunneling electron microscopy (TEM) image and dynamic light scattering (DLS) data, respectively, for metal-polymer hybrid nanoparticles prepared with a solvent stream of 6 mg/mL polystyrene-block-poly(vinylpyridine) (PS-b-PVP) in THF and a non-solvent stream of 0.45 mg/mL chloroauric acid (HAuCl₄) in H₂O. FIGS. 5C and 5D show a tunneling electron microscopy (TEM) image and dynamic light scattering (DLS) data, respectively, for metal-polymer hybrid nanoparticles prepared with a solvent stream of 1 mg/mL polystyrene-block-poly(vinylpyridine) (PS-b-PVP) in THF and a non-solvent stream of 0.15 mg/mL chloroauric acid (HAuCl₄) in H₂O.

### DETAILED DESCRIPTION

This application claims the benefit of U.S. Provisional Application No. 61/944,784, filed Feb. 26, 2014 and U.S. Provisional Application No. 62/042,515, filed Aug. 27, 2014, the specifications of which are hereby incorporated by reference.

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated.

The terms “particle”, “nanoparticle”, “colloid”, and “nanocolloid” are used interchangeably herein, unless another meaning is indicated by the context. The term “Janus” refers to a particle having two distinct surfaces, for example, having two surfaces of different polymers. The term “Janus” can also refer to a characteristic of such a particle or group of particles, such as “Janus morphology” or “Janus phase”.

Methods according to the invention apply to a broad range of polymer chemistries and cost effective processes to produce Janus particles. Processes according to the invention can produce bi- or tri-phasic, polymeric Janus particles which have distinct polymer chemistry in the phases and which have distinct surface chemistries on the faces.

Dissimilar polymers can be combined to create single colloids with phase-separated surfaces (Walther, A. et al., Janus discs, J. Am. Chem. Soc., 2007, 129, 6187-98). This can be accomplished by incorporating two or more distinct polymers into a single polymer chain to create block co-polymers that are then induced to assemble into Janus particles through a series of surface-based processing steps. However, multi-processing steps on a two-dimensional surface makes scalability non-trivial. Furthermore, particle size is fixed by the molecular weight (Mw) of the co-polymer (Walther, A. et al., Janus discs, J. Am. Chem. Sac., 2007, 129, 6187-98; Pochan, D. J. et al., Multicompartment and multigeometry nanoparticle assembly, Soft Matter 2011, 7, 2500).

The solution-based self-assembly of homopolymer molecules into nanoscale objects allows for the creation of Janus colloids with structural and compositional complexity (Jang, S. G. et al., Striped, ellipsoidal particles by controlled assembly of diblock copolymers, J. Am. Chem. Soc. 2013, 135, 6649-57; Yamashita, N. et al., Preparation of hemispherical particles by cleavage of micrometer-sized, spherical poly(methyl methacrylate)/polystyrene composite particle with Janus structure: effect of molecular weight, Colloid Polym. Sci, 2013, 292, 733-738; Higuchi, T. et al., Spontaneous formation of polymer nanoparticles with inner micro-phase separation structures, Soft Matter 2008, 4, 1302-1305; Kiyono, Y. et al., Preparation and Structural Investigation of PMMA-Polystyrene “Janus Beads” by Rapid Evaporation of an Ethyl Acetate Aqueous Emulsion, e-Journal Surf Sci. Nanotechnol. 2012, 10, 360-366). Such particles can be fabricated by dissolving multiple, chemically distinct polymers in a mutually favorable solvent and gradually altering the solubility character of the solution until the polymer molecules co-precipitate into self-organized structures. The final morphology adopted by the colloids via solution self-assembly can be unique to the particular processing conditions used, so that the range of architectures accessible to any one method is limited. This limitation on the range of accessible architectures constrains the applicability of these previous approaches. Also, the slow precipitation steps result in uncontrolled size distributions of the resulting particles. This is a major problem with the slow precipitation approaches, since control of particle size is essential in applications of these structured nanoparticles. The use of small amphiphilic surfactant molecules or polymeric stabilizers in the solution volume or a collection solution in which the polymers co-precipitate can mask the compositional heterogeneity and interfacial properties of the particle surface. The existing solution-based approaches usually operate under batch conditions with residence times of days or hours (Jang, S. G. et al., Striped, ellipsoidal particles by controlled assembly of diblock copolymers, J. Am. Chem. Soc. 2013, 135, 6649-57; Wang, Y. et al., Colloids with valence and specific directional bonding, Nature, 2012, 491, 51-5). For example, Higuchi and coworkers demonstrated the use of a slow solvent evaporation technique to form imperfect, micro-phase-separated nanoparticles from a solution containing polyisoprene and polystyrene. (Higuchi, T. et al., Spontaneous formation of polymer nanoparticles with inner micro-phase separation structures, Soft Matter, 2008, 4(6), 1302-1305). They were able to find, amongst the large array of other structures in the particles produced by the slow solvent evaporation, Janus particles. However, the Higuchi synthesis process had the following disadvantages: (1) it could not control the size of the assembled particle; (2) the slow precipitation could not produce nanoparticles with controlled stoichiometry (i.e., the least soluble polymer would precipitate first and produce nanoparticles that do not necessarily contain both polymers in a controlled ratio, so the process is not generalizable to arbitrary polymer pairs); and (3) the process took two days to slowly evaporate solvent from a 200 mL beaker, so that it was not scalable.

Therefore, a challenge remains to develop a continuous, scalable, and simple particle fabrication system that offers comprehensive control over multiple particle features such as particle size, surface domain size, and surface topology (Jang, S. G. et al., Striped, ellipsoidal particles by controlled assembly of diblock copolymers, J. Am. Chem. Soc. 2013, 135, 6649-57; Higuchi, T. et al., Spontaneous formation of polymer nanoparticles with inner micro-phase separation structures, Soft Matter 2008, 4, 1302-1305). Current strategies to produce polymer-polymer and/or polymer-inorganic Janus particles, including surface coating by vapor deposition, coating via Pickering emulsion, layer-by-layer self-assembly, biphasic electrified jetting, surface initiated polymerization, polymerization in microfluidic devices, and polymer phase separation, are not pathways to scalable technologies in which kilograms/day of material can be produced continuously.

A successful and scalable approach has two requirements: (1) a process must produce nano or microparticles of essentially uniform size; and (2) the polymeric contents of the nano or microparticle must spontaneously phase separate during the formation process to form a bi-, tri-, or multi-phasic structure.

The phase separation of polymer blends, a self-directed physical process capable of generating multi-domain structures at the nanoscale, can be used to fabricate structured multi-face particles (Sai, H. et al., Hierarchical porous polymer scaffolds from block copolymers, Science 2013, 341, 530-4). The complex structures associated with polymer phase separation may be transferred to colloids in a controllable manner by confining the volume and time scale in which polymer de-mixing takes place. The phase separation of dissimilar polymers precipitated from a common solvent via a confined impinging jet mixer can be induced through FNP. Polymer de-mixing is driven to occur within precipitating nanodroplets of polymer and solvent as the solvent rapidly exchanges on the order of milliseconds with a non-solvent during micro-mixing (Johnson, B. et al., Mechanism for Rapid Self-Assembly of Block Copolymer Nanoparticles, Phys. Rev. Lett. 2003, 91, 118302). The process unit, FNP, has advantages that render it a transformative route to Janus nanocolloids, including the following: i) a one-step and continuous process; ii) a room temperature and low energy process; and iii) proven scalability greater than 1400 kg/day of colloids.

With FNP, precursor polymers can be uniformly dispersed in a single phase solution and aggregate into particles with sufficiently uniform size. The FNP method can provide simultaneous control over particle size, surface functionality, and compositional anisotropy as the assembly process is scaled in the production of particles, such as Janus colloids assembled from two simple homopolymers. Tuning the molecular weight of the homopolymers and increasing the number of polymer components in the system can facilitate the formation of multi-faced and multi-lobal nanocolloids, respectively. Incompatible polymers with different properties can be self-assembled into nanocolloids with controllable surface topology by simultaneously reducing the timescale and solution volume over which they undergo self-assembly. FNP can create polymeric Janus particles with multi-phasic bulk and surface properties.

Alternatively, an emulsion of sufficiently uniform size can be created by mechanical dispersion. The emulsion comprises an internal dispersed phase containing the polymer components dissolved in a common solvent to afford a single phase fluid, and an external phase fluid in which the solvent phase is not completely miscible. The emulsion is stable under formation conditions. The solvent phase is then removed by an evaporation or extraction process, so that the polymers spontaneously phase separate during that removal or stripping process. This can create polymeric Janus particles with multi-phasic bulk and surface properties.

The nano or microparticles can be created without additional stabilizers. In that case the final Janus particle is in its final form. However, it may be necessary or desirable to process the particles with an added amphiphilic stabilizer to increase the stability of the particle or enable production at higher dispersed phase concentrations. In cases where a stabilizer is added it can be substantially removed from the surface by a subsequent step to unmask the particle, so that the two Janus (or three or more multi-face) surfaces display different surface chemistries. Examples of stabilizers that can be used include sulfonated alkyl surfactants, sodium dodecyl sulfate, ethoxylated sulfonate surfactants, cationic surfactants, amine oxide surfactants, zwitterionic surfactants, amphoteric surfactants, ethylene oxide surfactants based on alkyl ethers, ethylene oxide surfactants based on nonylphenols, surfactants based on sorbitan oleates, surfactants based on sugars such as glucose-based surfactants, polymeric surfactants such as polyethylene oxide-co-polybutylene oxide surfactants, such as the Pluronic or Pluroximer surfactants from BASF, polymeric stabilizers based on polyvinyl caprolactam and polycaprolactone, polymeric stabilizers based on partially hydrolyzed polyvinyl alcohol, polymeric stabilizers based on polyethylene oxide, natural products polymeric stabilizers based on substituted cellulose, such as hydroxypropyl cellulose, natural products polymeric stabilizers based on hydrophobically modified starches, lipids, and lecithin.

Without being bound by theory, the stability of a purely hydrophobic Janus particle may arise from the strong negative charge arising from hydroxyl adsorption (Beattie, J. K. et al., The surface of neat water is basic, Faraday discussions, 2009, 141, 31-39). Excessive salt is observed to precipitate the particles, which is consistent with this view.

Molecular weight ranges of polymers useful for forming Janus particles range from the lowest molecular weight that creates macroscopic phase separation, for example, 800 Da (Dalton or g/mol) for polystyrene molecules in a blend with polyisoprene, up to 10⁵ Da molecular weight or up to 10⁷ Da molecular weight. For examples, polymers having molecular weights ranging from about 800 Da, 1 kDa, 3 kDa, 10 kDa, 30 kDa, 100 kDa, 300 kDa, 1000 kDa, or 3000 kDa, to about 1 kDa, 3 kDa, 10 kDa, 30 kDa, 100 kDa, 300 kDa, 1000 kDa, 3000 kDa, or 10,000 kDa can be used. Macroscopic, bulk phase separation can be determined by any classical experimental technique, including imaging the final Janus particle.

The Janus formation process is widely applicable, and is not limited to specific polymer chemistry. The following polymers with purely hydrophobic terminal units, with hydroxyl (OH), and with carboxyl (COOH) units have been formed into Janus particles. Janus particles have been formed from polymers over the molecular weight range 9,000 to 1,000,000. Examples of Janus particles formed are provided in Table 1.

TABLE 1

| | | MW | |
|---|---|---|---|
| Polymer | Name | (kg/mol) | End functionalization |
| 1 | Atactic Polystyrene | 16.5 | None (hydrogen terminated) |
| 2 | Atactic Polystyrene | 1500 | None (hydrogen terminated) |
| 3 | Carboxy terminated Polystyrene | 16.5 | carboxylic acid |
| 4 | hydroxy-terminated Polystyrene | 16 | hydroxyl group |
| 5 | Polyisoprene (1,4 addition) | 11 | None (hydrogen terminated) |
| 6 | Polyisoprene (1,4 addition) | 1000 | None (hydrogen terminated) |
| 7 | Polybutadiene | 9.1 | None (hydrogen terminated) |
| 8 | Polybutadiene(1,4 addition) | 18 | None (hydrogen terminated) |
| 9 | Hydroxy-terminated | 12.5 | hydroxyl group |
| | Polybutadiene | | |
| 10 | Polyvinyl cyclohexane | 25 | None (hydrogen terminated) |

Janus Particle Polymer Pairings:
1 & 5
2 & 6
4 & 5
4 & 9
3 & 5
3 & 9
10 & 8
Tricomponent Polymer Groupings:
10, 8, 1
10, 7, 1

Because Janus structures can be made from polymers with a wide range of terminal functionality, the Janus particles can be reacted after formation to impart desirable surface properties on the Janus faces. For example, the COOH or OH can be reacted with amine groups or the OH with acid chlorides to attach more hydrophilic entities on one Janus face. This can enhance the hydrophobicity/hydrophilicity difference between the two faces and can enhance the interfacial stabilization properties of the construct. A range of other surface modifications are possible, and they can be designed to impart a variety of Janus properties.

### Flash NanoPrecipitation

Flash NanoPrecipitation (FNP) can be used for the production of organic and organic/inorganic nanoparticles. The mean particle diameter of these nanoparticles can be in the range of from 30 to 2000 nm, for example, from about 50 to 800 nm. For example, the mean particle diameter of these nanoparticles can be from about 10, 20, 30, 50, 60, 100, 200, 300, 500, 800, 1000, 1200, 1500, 2000, 4000, 5000, 6000, or 10,000 nm to about 20, 30, 50, 60, 100, 200, 300, 500, 800, 1000, 1200, 1500, 2000, 4000, 5000, 6000, 10,000, or 20,000 nm. FNP can form particles of narrow size distribution. For example, of the nanoparticles formed, at least 90% can have a diameter less than 800 nm, and at most 10% can have a diameter less than 50 nm. For example, of the nanoparticles formed, at least 90% can have a diameter less than 50,000, 20,000, 10,000, 6000, 5000, 4000, 2000, 1000, 800, 500, 200, 100, 60, 50, 30, 20, or 10 nm, and at most 10% can have a diameter less than 20,000, 10,000, 6000, 5000, 4000, 2000, 1000, 800, 500, 200, 100, 60, 50, 30, 20, 10, or 5 nm.

The FNP process uses micromixing geometries to mix an incoming, miscible solvent stream in which a polymer is dissolved (so that it can also be termed a polymer solution stream) with a non-solvent stream to produce supersaturation levels as high as 10,000 with mixing times of about 1.5 ms. For example, supersaturation levels can range from about 100, 300, 1000, 3000, 10,000, 30,000, 100,000, or 300,000 to about 300, 1000, 3000, 10,000, 30,000, 100,000, 300,000, or 1,000,000, and mixing times can range from about 0.01, 0.03, 0.1, 0.3, 1, 1.5, 3, 10, 15, 30, 100, or 300 ms to about 0.03, 0.1, 0.3, 1, 1.5, 3, 10, 15, 30, 100, 300, or 1000 ms. It is desirable that these mixing times are shorter than the nucleation and growth times of nanoparticle assembly, so that the size of the nanoparticles formed is constrained. The solvent stream and non-solvent stream can be further mixed with a collection solution, for example, a collection solution that includes a stabilizer such as an amphiphilic surfactant molecule. Nanoparticles can be formed for a variety of pharmaceutical compound, imaging agent, security ink, and drug targeting applications (Johnson, B. K. et al., Chemical processing and mieromixing in confined impinging jets, AIChE J. September 2003, 49(9), 2264-2282; Johnson B. K. et al., Mechanism for rapid self-assembly of block copolymer nanoparticles, Phys. Rev. Lett. Sep. 12, 2003, 91(11); Johnson, B. K. et al., Flash NanoPrecipitation of organic actives and block copolymers using a confined impinging jets mixer, Australian J. Chem. 2003, 56(10), 1021-1024; Johnson, B. K. et al., Nanoprecipitation of organic actives using mixing and block copolymer stabilization, Abstracts of Papers of the American Chemical Society September 2003, 226, U487-U487; Johnson B. K. et al., Engineering the direct precipitation of stabilized organic and block copolymer nanoparticles as unique composites, Abstracts of Papers of the American Chemical Society September 2003, 226, U527-U527; Johnson, B. K. et al., Nanoprecipitation of pharmaceuticals using mixing and block copolymer stabilization, Polymeric Drug Delivery II: Polymeric Matrices and Drug Particle Engineering 2006, 924, 278-291; Ansell, S. M. et al., Modulating the therapeutic activity of nanoparticle delivered paclitaxel by manipulating the hydrophobicity of prodrug conjugates, J. Med. Chem. June 2008, 51(11), 3288-3296; Gindy, M. E. et al. Preparation of Poly(ethylene glycol) Protected Nanoparticles with Variable Bioconjugate Ligand Density, Biomacromolecules October 2008, 9(10), 2705-2711; Gindy, M. E. et al., Composite block copolymer stabilized nanoparticles: Simultaneous encapsulation of organic actives and inorganic nanostructures, Langmuir January 2008, 24(1), 83-90; Akbulut M. et al., Generic Method of Preparing Multifunctional Fluorescent Nanoparticles Using Flash NanoPrecipitation, Adv. Funct. Mater. 2009, 19, 1-8; Budijono, S. J. et al., Exploration of Nanoparticle Block Copolymer Surface Coverage on Nanoparticles, Colloids and Surfaces A-Physicochemical and Engineering Aspects, 2010; Budijono, S. J. et al., Synthesis of Stable Block-Copolymer-Protected NaYF₄:Yb³⁺, Er³⁺ Up-Converting Phosphor Nanoparticles, Chem. Mat. 2010, 22(2), 311-318; D'Addio, S M. et al., Novel Method for Concentrating and Drying Polymeric Nanoparticles: Hydrogen Bonding Coacervate Precipitation, Molecular Pharmaceutics March-April 2010, 7(2), 557-564; Kumar, V. et al., Fluorescent Polymeric Nanoparticles: Aggregation and Phase Behavior of Pyrene and Amphotericin B Molecules in Nanoparticle Cores, Small December 2010, 6(24), 2907-2914; Kumar, V. et al., Stabilization of the Nitric Oxide (NO) Prodrugs and Anticancer Leads, PABAJNO and Double JS-K, through Incorporation into PEG-Protected Nanoparticles, Molecular Pharmaceutics January-February 2010, 7(1), 291-298; D'Addio, S. M. et al., Controlling drug nanoparticle formation by rapid precipitation, Adv. Drug Delivery Rev. May 2011, 63(6), 417-426; Kumar, V. et al., Fluorescent Polymeric Nanoparticles: Aggregation and Phase Behavior of Pyrene and Amphotericin B Molecules in Nanoparticle Cores, Small December 2011, 6(24), 2907-2914; Shan, J. N. et al., Pegylated Composite Nanoparticles Containing Upconverting Phosphors and meso-Tetraphenyl porphine (TPP) for Photodynamic Therapy, Adv. Functional Materials July 2011, 21(13), 2488-2495; Shen, H. et al., Self-assembling process of flash nanoprecipitation in a multi-inlet vortex mixer to produce drug-loaded polymeric nanoparticles, J. Nanoparticle Res. September 2011, 13(9), 4109-4120; Zhang, S. Y. et al., Photocrosslinking the polystyrene core of block-copolymer nanoparticles, Polym. Chem. March 2011, 2(3), 665-671; Zhang, S. Y. et al., Block Copolymer Nanoparticles as Nanobeads for the Polymerase Chain Reaction, Nano Lett. April 2011, 11(4), 1723-1726; D'Addio, S. M. et al., Constant size, variable density aerosol particles by ultrasonic spray freeze drying, Int'l J. Pharmaceutics May 2012, 427(2), 185-191; D'Addio, S. M. et al., Effects of block copolymer properties on nanocarrier protection from in vivo clearance, J. Controlled Release August 2012, 162(1), 208-217; D'Addio, S. M. et al., Optimization of cell receptor-specific targeting through multivalent surface decoration of polymeric nanocarriers, J. Controlled Release May 2013, 168(1), 41-49; Figueroa, C. E. et al., Effervescent redispersion of lyophilized polymeric nanoparticles, Therapeutic Delivery 2013, 4(2), 177-190; Figueroa, C. E. et al., Highly loaded nanoparticulate formulation of progesterone for emergency traumatic brain injury treatment, Therapeutic Delivery 2013, 3(11), 1269-1279; Pinkerton, N. M. et al., Formation of Stable Nanocarriers by in Situ Ion Pairing during Block-Copolymer-Directed Rapid Precipitation, Mol. Pharmaceutics 2013, 10, 319-328; Pinkerton, N. M. et al., Gelation Chemistries for the Encapsulation of Nanoparticles in Composite Gel Microparticles for Lung Imaging and Drug Delivery, Biomacromolecules 2013; DOI: 10.1021/bm4015232). Flash NanoPrecipitation can be used with stabilizing block copolymers to produce nanoparticles. Alternatively, FNP can be used for the production of polystyrene particles without an added stabilizer or amphiphilic copolymer. Nanoparticles over the size range of 60 to 200 nm with polydispersities comparable to those produced by emulsion polymerization were obtained using only electrostatic stabilization.

FNP overcomes the limitations of previous approaches that did not control the size of the assembled nanoparticles, were unable to produce nanoparticles with controlled stoichiometry, and were slow and not scalable. With FNP, nanoparticle size can be controlled. Rapid micromixing to a uniform high supersaturation produces diffusion limited aggregation, and the aggregating solutes or polymers “stick” randomly to each other, so that each particle contains the stoichiometric ratio of solutes that are introduced into the FNP micromixer. Although the process is random, because each nanoparticle contains polymer chains on the order of 50,000 Da molecular weight, the variance in concentration between particles is small. The FNP process takes on the order of 15 ms for particle formation. The FNP process has been scaled to 1400 kg/day by BASF.

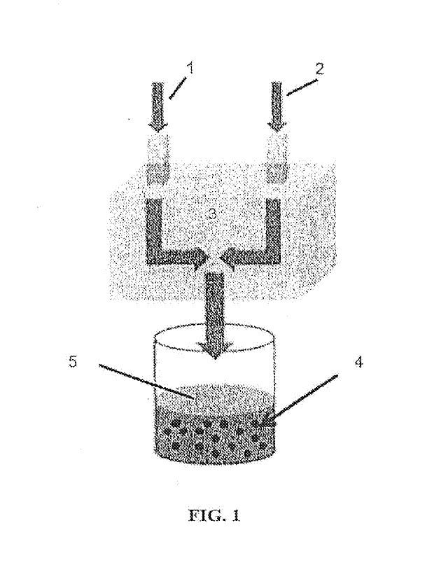

Thus, FNP is a room temperature, low energy, one-step, rapid, and continuous route to produce polymer-polymer Janus nanoparticles. A schematic of the FNP process is illustrated in FIG. 1. The mixing occurs in a central cavity 3 fed by two incoming streams 1 and 2 that are high velocity linear jets of fluid. The one stream 1 contains the polymers dissolved in a solvent. The other stream 2 is of a non-solvent for the polymer. The compositions and ratios of the streams are chosen so that after mixing in the central cavity 3, the polymers are no longer dissolved and rapid precipitation occurs (Johnson, B. K. et al., AIChE J. 2003, 49, 2264; Johnson, B. K. et al, Phys. Rev. Lett. 2003, 91; Johnson, B. K. et al., Aust. J. Chem. 2003, 56, 1021; Pustulka, K. M. et al., Mol. Pharmaceutics, 2013, 10, 4367). The nanoparticles formed 4 can be collected in a collection solution 5. Different mixing geometries can be used in this process, as long as the selected mixing geometry selected produces rapid micromixing to control precipitation (Burke, P. A. et al., international patent application PCT/US2011/031540 and U.S. published patent application US20130037977). The polymer solution rapidly mixes with the non-solvent for a few milliseconds to induce self-assembly of the polymers into kinetically frozen nanoparticles. When used to form polymeric Janus particles two polymers may be dissolved in the solvent (e.g., an organic solvent) of stream 1. However, other hydrophobic components such as small molecule drugs, imaging agents, particles, and therapeutic agents can be successfully encapsulated into polymeric nanoparticles by FNP (Shan, J. et al, Adv. Funct. Mater. 2011, 21, 2488.; Kumar, V. et al., Small 2010, 6, 2907; Pinkerton, N. M. et al., Biomacromolecules 2014, 15, 252). A wide range of solvents and non-solvents that are miscible can be used in the process. Solvents include materials in which the polymer components are soluble. The solvent is miscible with the non-solvent. Nonsolvents include materials in which the polymer components are not soluble or are only sparingly soluble. For example, the solvent can be a non-aqueous solvent, such as an organic solvent or a low polarity solvent, and the non-solvent can be water, a predominantly aqueous phase, or a high polarity solvent. Alternatively, the solvent can be water or a high polarity solvent (for example, if the polymer to be dissolved is a hydrophilic polymer) and the non-solvent can be a non-aqueous solvent or a low polarity solvent. Alternatively, the solvent and the non-solvent can be selected from two different non-aqueous solvents. The solvent or the non-solvent can be polar or nonpolar (or have an intermediate polarity) and can be protic or aprotic. Examples of materials that can be used as solvents or non-solvents include water, alcohols, such as methanol, ethanol, isopropanol (2-propanol), and n-propanol (1-propanol), carboxylic acids, such as formic acid, acetic acid, propanoic acid (propionic acid), butyric acid, furans, such as tetrahydrofuran (THF), dioxane, 1,4-dioxane, furfuryl alcohol, ketones, such as acetone and methyl ethyl ketone (MEK), other water-miscible solvents, such as acetaldehyde, ethylene glycol, propanediol, propylene glycol (propane-1,2-diol), 1,3-propanediol, butanediol, 1,2-butanediol, 1,3-butanediol, 1,4-butanediol, pentanediol, 1,5-pentanediol, 2-butoxyethanol, glycerol, triethylene glycol, dimethyl sulfoxide (DMSO), ethylamine, diethanolamine, diethylenetriamine, methyl diethanolamine, dimethylformamide (DMF), and pyridine, acetonitrile, methyl isocyanide, esters, such as methyl acetate and ethyl acetate, ethers, such as diethyl ether and dimethoxyethane, carbon disulfide, halogenated organics, such as carbon tetrachloride, alkanes, such as heptane, alkenes, such as hexene, cycloalkanes, such as cyclohexanc, aromatic hydrocarbons, such as toluene, other organic and inorganic materials, and mixtures of these. A further description of solvent compositions useful for processing by FNP has been presented in B. K. Johnson, R. K. Prud'homme, US Patent Application Pub. US 2012/0171254 A1, Jul. 5, 2012.

FNP is useful in producing homogenous nanoparticles of various polymers including polystyrene, polymethylmethacrylate, and polycaprolactone with controlled diameters and narrow polydispersity indexes (PDIs) (Kumar V. et al., Preparation of lipid nanoparticles: Google Patents, 2013 (EP2558074)). Neither premade nanoparticles nor immobilization steps are required for the FNP process. By simply adjusting the initial polymer concentrations, it is possible to tune the anisotropy of the Janus nanoparticles. Hybrid polymer-inorganic Janus nanoparticles can be made by the FNP process. FNP has been previously described in the following patent documents, which are incorporated by reference into this submission in their entirety:

Preparation of Lipid Nanoparticles, M. Cindy, et al., US Patent Publication, US20130037977 A1, PCT/US2011/031540, publication date Feb. 14, 2013;

A high-loading nanoparticle-based formulation for water-insoluble steroids, C. Figureroa et al., Patent Publication, WO2013063279 A1, PCT/US2012/061945, publication date May 2, 2013;

Particulate constructs for release of active agents, L. D. Mayer et al., Patent Publication US20130336915 A1, Publication date Dec. 19, 2013; and

Process and Apparatuses for Preparing Nanoparticle Compositions with Amphiphilic Copolymers and Their Use, B. K. Johnson et al., US Patent Application Pub., US 2012/0171254 A1, Jul. 5, 2012.

The production of single component polymer nanoparticles by FNP has been described in Zhang, C. et al., Flash nanoprecipitation of polystyrene nanoparticles, Soft Matter 2012, 8(1), 86-93, which is also incorporated herein by reference in its entirety.

The FNP process requires adequate micromixing, which has been described in the patents above. FNP requires that the polymers or inorganic colloids of interest be mutually soluble in a common organic process solvent which is miscible with the non-solvent stream. Water or an aqueous solution can be used as the non-solvent stream and a water-miscible organic solvent can be used as the process solvent stream. With the polymer additives the convergence of the two streams produces a dispersed Janus nanoparticle dispersion in the mixed solvent phase.

The FNP process may be run without a stabilizer additive, so that the process solvent contains the polymers and/or colloids of interest without an amphiphilic stabilizer. Alternatively, amphiphilic stabilizers may be added to either the process solvent phase or the non-solvent phase. It is also possible to reverse the solvent polarity and to precipitate water soluble Janus particles in a non-aqueous non-solvent phase.

Particles may be produced by the FNP process to have, for example, diameters between 10 nm and 4000 nm, between 20 nm and 1000 nm, or between 50 nm and 800 nm. The sizes are the intensity weighted average size determined by dynamic light scattering. Such measurements can be conducted in a Malvern Nanosizer dynamic light scattering (DLS) instrument. The size reported by dynamic light scattering is the intensity weighted diameter, which is used herein to report sizes of the particles produced by the Flash NanoPrecipitation process. The breadth of distribution of the particle diameters can be characterized by values such as the Di90, the intensity-weighted diameter where 90% of the particles have a lesser diameter, the Di50, the intensity-weighted diameter where 50% of the particles have a lesser diameter, and the Di10, the intensity-weighted diameter where 10% of the particles have a lesser diameter. For example, to define a minimum narrowness of distribution of particle diameters, it can be specified that at least 90% of the particles have a diameter less than a nominal Di90 value and that at most 10% of the nanoparticles formed have a diameter less than a nominal Di10 value, or that 80% of the nanoparticles have a diameter greater than or equal to the nominal Di10 value and less than the nominal Di90 value. Alternatively, the Span can be defined as the difference between the Di90 and Di10 values divided by the Di50 value, that is, Span=(Di90-Di10)/Di50. A smaller Span indicates a more narrow distribution of particle sizes, with a Span of zero indicating a monodisperse distribution (i.e., all particles have the same size. The Di10, Di50, and Di90 values are determined from the intensity weighted distribution that is obtained from the dynamic light scattering measurement. These values can be calculated on a mass-weighted basis using standard conversions from intensity-to mass-weighted distributions.

### Nanoparticle Formation Through Emulsions Formed by Mechanical Dispersion

In the alternative process, an emulsion can be formed by mechanical agitation using, for example, impellers, rotor-stator mixers, porous plate, or micro-structured plate emulsifiers. For example, the use of mechanical disruption in a uniform shear field with control of internal and external viscosity ratios has been described by Bibbette and used by Pinkerton (Pinkerton, N. M. et al., Formation of Stable Nanocarriers by in Situ Ion Pairing during Block-Copolymer-Directed Rapid Precipitation, Mol. Pharmaceutics 2013, 10, 319-328).

Dilution of the polymer by a solvent in the internal phase achieves miscibility of the polymer species. The solvent phase can be removed from emulsion drops by a “stripping” process. Stripping can be achieved by any means. Two processes are direct evaporation and extraction. In direct evaporation the solvent phase has some limited solubility in the external phase and this solvent transfers from the drop interior, through the external phase, to the external atmosphere where it is removed. This evaporation step can be slow and is dependent on the surface area available to remove the solvent from the external phase.

In extraction an additional component is added to the external phase once the emulsion has been fully formed and stabilized. The added component is one that increases the solubility of the internal solvent into the external fluid phase. The increased solubility “strips” the internal solvent from the emulsion drops and transfers it to the external phase.

By either stripping process the increase in polymer concentration inside the emulsion drop creates sufficient polymer:polymer interactions, so that phase separation is achieved and the Janus structure is established. For example, particles can be produced that are between 20 nm and 20,000 nm, between 20 nm and 6000 nm, between 30 nm and 1000 nm, or between 50 nm and 800 nm.

Stabilizers and their Removal

Stabilizers that have been incorporated into either the FNP process or the emulsion by a mechanical dispersion process may need to be substantially removed from the final Janus formulation, depending on the application of the particles.

In a first approach, if the stabilizers have a solubility in the external phase of greater than, for example, 10⁻³ wt %, then they can be removed by solvent exchange and diffusion. This can be done by batch dialysis, by tangential flow ultrafiltration, by centrifugation and decanting, or other processes for removing soluble impurities from particulate suspensions. Once the amphiphilic stabilizer is removed the intrinsic properties of the polymers comprising the Janus core will be exposed.

A second approach involves specific complexation of the surfactant to remove it from the particle surface. One such example is the complexation of surfactants, notably sodium dodecyl sulfate (SDS), using cyclodextrins. The binding constant of SDS to cyclodextrin has a higher affinity than binding to the particle surface. Thus, the surfactant can be removed from the surface. This process has been described for the removal of SDS from hydrophopic associative polymers, and has been used in the synthesis and purification of copolymers using cyclodextrins. The SDS:cyclodextrin complex is stable in the particle dispersion, but it may be desirable to remove the soluble SDS:cyclodextrin complex by one of the methods presented above. SDS is a representative interfacial stabilizer for either FNP or emulsion processing, but other surfactants strongly binding with cyclodextrin can also be used.

In some FNP and emulsion processes it may be desirable to use larger amphiphilic polymers, block copolymers, or surfactants whose solubility is so low that they cannot be removed by the processes described above. For this, a third approach is to use cleavable amphiphilic stabilizers in which the hydrophilic moiety in the stabilizer is attached to the hydrophobic moiety by a linker that may be broken or cleaved. The result is that the hydrophobic moiety is left on the surface and the hydrophilic moiety is dispersed in the external aqueous phase. For an external hydrophobic solvent phase the system is reversed. Cleavable linkages may be esters, orthoesters, ketal, disulfides, or other groups well known in the field that are cleaved by hydrolysis, redox reactions, exchange reactions, enzymatic attack, or other chemical or biochemical reactions that can be initiated by changes in pH, redox conditions, or the addition of catalytic species. In some cases, this third approach using cleavable amphiphilic stabilizers may be desirable to produce nanoparticles that do not contain amphiphilic components in the final formulation, the cleaving process renders the stabilizer no longer amphiphilic.

If the amphiphilic anchoring species is dilute enough on the particle surface, then its presence on the surface will not alter the desirable Janus surface properties.

In another embodiment, when the amphiphilic anchoring block is high enough in molecular weight, for example, above 900 Daltons, it can become part of the Janus phase. For example, an FNP or emulsion process can be conducted with an amphiphilic block copolymer having the hydrophobic block of the same type as the material composing the Janus particle. After Janus particle formation the amphiphilic anchoring block is cleaved removing the soluble portion from the particle surface, while leaving the anchoring block anchored in the polymer matrix. Two or more amphiphilic stabilizers can be used, each with the hydrophobic block being that of one of the polymer phases in the Janus core. The amphiphilic stabilizers will partition on the particle surface, driven by the enthalpic energy of phase separation, which drives the phase separation of the Janus core. Once the soluble components are cleaved the Janus particle will have separate phases which now include the component from the amphiphilic stabilizer. The ratio of amphiphilic stabilizers is adjusted to approximately reflect the volume ratio of the Janus particle core. For example, if the Janus particle is to have a 50:50 ratio of two polymers, the stabilizers should be used in approximately a 50:50 ratio. The exact ratio depends on both the size of the hydrophobic and the hydrophilic blocks to create an optimal surface area ratio.

Without intending to be bound be theory, Flory-Huggins theory can be applied to understand the micro-phase separation in nanoparticle (NP) cores that results in the formation of Janus particles. The Chi parameter, χ, characterizes the strength of interactions between dissimilar polymers and χN parameterizes the total interaction energy of a polymer with N statistical segments. Chi values and the number of monomers per statistical segment are known for most polymers.

Without intending to be bound by theory, the interfacial energy of the two or more polymers can play a role in the Janus structure obtained. From an argument of the total free energy of a Janus particle, it is expected that Janus structure will arise if the absolute value of the surface energy difference between the external (water) phase and polymer A and B is greater than the interfacial energy between components A and B:

γ_{AB}<1.46|(γ_{BW}−γ_{AW})|

If this equality does not hold then a core-shell morphology can be formed. Thus, the formation of a core-shell versus a Janus morphology is an important structural feature that can be controlled.

Janus particles with some fraction of the polymer composition being a block copolymer that has desirable phase behavior properties with the two or three other polymer components can be formed. This enables Janus particle formation by tuning the interfacial energy between the major homopolymer components.

### Isolation of Janus Particles

Post processing steps can be applied to the particle phases to concentrate the Janus particles, remove residual process solvent, change the process solvent, or change the non-solvent. Concentration can be effected by ultrafiltration, selective flocculation, for example, as described by D'Addio, S. M. et al., Novel Method for Concentrating and Drying Polymeric Nanoparticles: Hydrogen Eionding Coacervate Precipitation. Molecular Pharmaceutics March-April 2010, 7(2), 557-564, centrifugation, freeze drying, spray drying, or tray or drum drying. Excipients may be added during the drying or concentrating phases to minimize Janus particle aggregation, or to enhance redispersion. For example, polyethylene glycol from 1000 to 20,000 molecular weight can be used as an excipient.

### Cerberus Particles

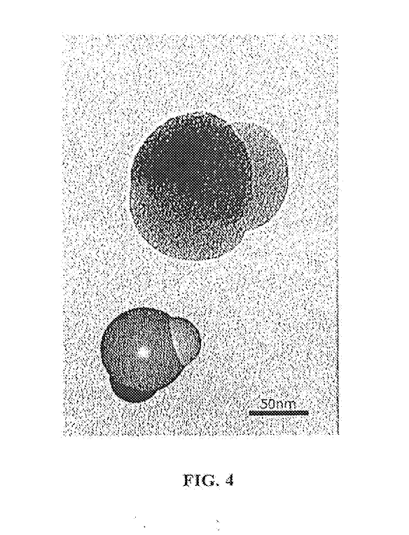

By introducing more than two homopolymers into the FNP system, multi-faced nanocolloids with more than two faces can be produced. For example, by introducing three polymers, tri-lobal nanocolloids, termed Cerberus particles, can be formed. FIG. 4 shows a three-component Janus particle formed from an equal mixture of polyvinylcyclohexane (PVCH, 25 kDa), polybutadiene (PB, 18 kDa), and polystyrene (PS, 16 kDa).

The ability to create multi-faced nanocolloids including Janus and Cerberus particles from two or more homopolymers not only attests to the versatility of the FNP process, but affords opportunities to construct more sophisticated multi-surface colloids in the future. Through PISA-FNP, colloidal size, anisotropy, and surface functionality can be independently controlled, providing a rapid, solution-based strategy for the formation of soft multi-faced nanocolloids. The simplicity and scalability of the process, furthermore, provides a platform for Janus particle production commensurate with current technological interest.

### Applications of Janus and Cerberus Particles

Janus and Cerberus particles produced by the processes described above can be useful in applications such as the following:

stabilization of interfaces in liquid:liquid dispersions;

stabilization of interfaces in gas:liquid foams and dispersions;

stabilization of interfaces in liquid:solid dispersions, such as in pigment stabilization;

stabilization of waxes and asphaltenes in oil and fuel processing;

formation of self-assembling structures based on interactions with one of the Janus surfaces; and

formation of a coating where one face of the Janus particle interacts preferentially with a solid surface and the other face of the Janus particle creates a second surface property that is desirable, such as color, texture, adhesion, anti-biofouling, tactile feel, reflection, hardness, softness, or bonding to a second surface coating.

### Stabilized Homopolymer Particles

A method according to the invention includes making stabilized homopolymer particles. The stabilized homopolymer may have metal formed thereon. The stabilized homopolymer may lack metal formed thereon. The methods of making stabilized homopolymers may include flash nanoprecipitation. According to one embodiment a first fluid stream including a homopolymer (e.g., selected from polystyrene, poly(methyl methacrylate), polycaprolactones, polylactides, polyamides, polysulfones, polyimides, and other polymers) and a solvent therefor is rapidly mixed with a second fluid stream that includes a non-solvent for the homopolymer.

The method of making stabilized homopolymer particles may include merging the mixing streams into a water reservoir that includes a stabilizer (e.g., anionic surfactant) such as sodium dodecyl benzene sulfonate. The homopolymer particles may include only a single polymer. Alternatively, the “homopolymer” particles may also include a small amount of comonomer, in which case they can also be referred to as “near homopolymer” particles. For example, the “homopolymer” or “near homopolymer” may have a small amount (e.g., 5% by weight, or up to about 1%, 2%, 3%, 5%, 10%, or 20% by weight) of acrylic acid comonomer.

The method of making stabilized homopolymer particles may employ a single “homopolymer” (having up to about 5% co-monomer, or up to about 1%, 2%, 3%, 5%, 10%, or 20% by weight co-monomer) and no second polymer in the feed stream. This method enables making homopolymer particles with stabilizers without comonomer, or only a small amount of comonomer.

### Organic-Inorganic Janus Particles

A Janus structure may contain two or more polymer phases and an inorganic material may be imbedded in one of the polymer phases, or it may be deposited or reacted on the surface of one of the Janus faces, or it may be reacted into the bulk of one of the Janus polymer phases. The reaction steps can occur during or after the particle formation step.

The physical self-assembly of hybrid inorganic/polymer nanocomposites previously required pre-synthesized nanoparticles with well-defined surface chemistry. However, the flash nanoprecipitation process of the current invention allows for the self-assembly of hybrid organic-inorganic nanoparticles in one step. Slower emulsion stripping processes can also be practiced if the stabilizing agent can be removed, or does not interfere with the desired Janus functionality of the final particle. FNP can fabricate nanoparticles composed of inorganic metals and organic polymers, and the process may be further extended to the fabrication of hybrid particles containing other inorganic nanomaterials such as crystals in addition to polymers. Organic-inorganic particles can also be formed using the emulsion stripping process described above. Spherical polyelectrolyte brushes (SPBs) can be used for the generation and stabilization of metallic nanoparticles. These SPBs can consist of a solid polystyrene (PS) core onto which long anionic or cationic polyelectrolyte chains are grafted. The dense layer of polyelectrolyte chains confined on the surface of the core particles can be used to immobilize metal ions. Reduction of these immobilized metal ions with a reducing agent, such as sodium borohydride (NaBH₄), can lead to nanoparticles of the respective metal. Other reducing agents that can be used include lithium aluminum hydride (LiAlH₄), compounds containing the Sn²⁺ ion, such as tin(II)chloride (SnCl₂), compounds containing the Fe²⁺ ion, such as iron(II)sulfate (FeSO₄), oxalic acid, formic acid, ascorbic acid, sulfite compounds, phosphites, hydrophosphites, and phosphorous acid, dithiothreitol (DTT), and tris(2-carboxyethyl)phosphine HCl (TCEP), carbon, carbon monoxide (CO), diisobutylaluminum hydride (DIBAL-H), Lindlar catalyst, sodium amalgam, zinc amalgam (Clemmensen reduction), diborane, hydrazine (Wolff-Kishner reduction), and nascent (atomic) hydrogen.

The whole process can involve multiple steps of synthesis of core-shell polymeric nanoparticles, adsorption of metal ions, and reduction and stabilization of metal nanoparticles.

A one-step synergistic preparation of metal nanoparticles within spherical polymer nanoparticles through Flash NanoPrecipitation (FNP) was carried out. FNP can generate monodisperse polystyrene nanoparticles and illustrated that the sizes of PS nanoparticles can be fine-tuned between 30 and 150 nm by changing the polymer and/or electrolyte concentration (Zhang, C. et al., Soft Matter 2012, 8, 86; Chung, J. W., J. Colloid interface Sci. 2013, 396, 16). Advantages of FNP are that it is a three-component process (involving only bulk polymer, a solvent, and a non-solvent) with low residence time, continuous processing, low energy consumption, and high reproducibility.

Polystyrene-block-poly(vinylpyridine) (PS-b-PVP) in THF, aqueous metal ion salts, and reducing agent solutions are employed as polymer stream, non-solvent stream and collection solution, respectively. Uniform metal nanoparticles grown on polymer nanospheres are obtained through a one-step FNP process. The particle size and metal nanoparticle loading amount can be tuned by changing the preparation parameters (e.g., feeding amount, feeding speed, and mixing rate).

FNP can fabricate nanoparticles composed of inorganic metals and organic polymers, and the process may be further extended to the fabrication of hybrid particles containing other inorganic nanomaterials such as crystals in addition to polymers. The process is applicable to many metals and mixtures of metals. A block copolymer that can be used on one face of the Janus particle can be have specific interactions with the inorganic or metal ion reactant. The inorganic may be deposited as a metal, or as a metal or mixed metal oxide. Many reducing agents may be employed in the reduction step, and may be introduced during the FNP process or, in some cases, added later to the reaction bath. Other reactions to deposit oxides, electrode-less electrochemical deposition, or inorganic reactions between the polymer surface and the inorganic precursor can be conducted. In comparison to existing processes, the one-step FNP technique is a single-step, low energy, continuous, and rapid process.

The FNP route to metal-polymer nanoparticles is a one-step process with a short processing time. This approach allows for the development of a range of hybrid nanoparticles with varying structural features that are difficult to achieve by conventional multi-step synthetic approaches. Examples of metals include gold (Au), platinum (Pt), silver (Ag), palladium (Pd), copper (Cu), cobalt (Co), and iron (Fe). Other metals and combinations of these can be used. For example, metals and combinations of metals that have catalytic properties can be used. Block copolymers in which one block is composed of a polyelectrolyte (e.g., PS-co-PVP) are suitable.

Metal-polymer nanoparticles are useful in nanotechnology applications, including single nanoreactors, catalyst supports, adsorbents, drug delivery vehicles, medical imaging agents, emulsion stabilization and chemical reactivity agents for emulsion-based reaction processes, large-scale composite nanomaterials, sensors and electronic devices (Xu, P. et al., Chem. Soc. Rev. 2014, 1349). Thus, metal-polymer nanoparticles can find application in the waste water treatment, battery, oil industry, and medical sectors.

Two challenges with this technology are controlling the stability of the particles in aqueous solution and the distribution of particle sizes that broaden as the concentration of polymers in the input stream is increased. Both of these challenges, though, may be overcome by optimizing the particle processing conditions or by incorporating stabilizing agents such as surfactants in the nanoparticle fabrication process.

The process according to the invention of producing metal-polymer nanoparticles is facile and rapid compared to other approaches. Currently, metal-polymer nanoparticles are not commercially available. Previously, metal-polymer nanoparticles could not be made in a scalable, cost-efficient way.

### Example 1

### Formation of Polystyrene (PS): Polyisoprene (PI) Janus Particle with Amphiphilic Block Copolymer

The FNP process can be used. Along with polystyrene (PS) and polyisoprene (PI) homopolymers, two amphiphilic block copolymers can added to the THY stream. For example, the polymers can be polystyrene-block-polyethylene oxide (PS-b-PEO) (P9669B1-EOS cleavable from Polymer Source, Canada) and a similar PI-b-PEO at a ratio of 50:50 based on the mass of the PEO block. FNP on the mixture can produce nanoparticles that are stable and for which the solvent can be removed by dialysis. To the resulting nanoparticle sample hydrochloric acid (HCl) can be added to produce a pH of 1.5. After 24 hours the sample can be dialyzed against distilled water to obtain a Janus particle dispersion, essentially free of polyethylene oxide (PEO), with a surface chemistry of pure PI and PS.

### Example 2

### Polystyrene-Polyisoprene Janus Particles

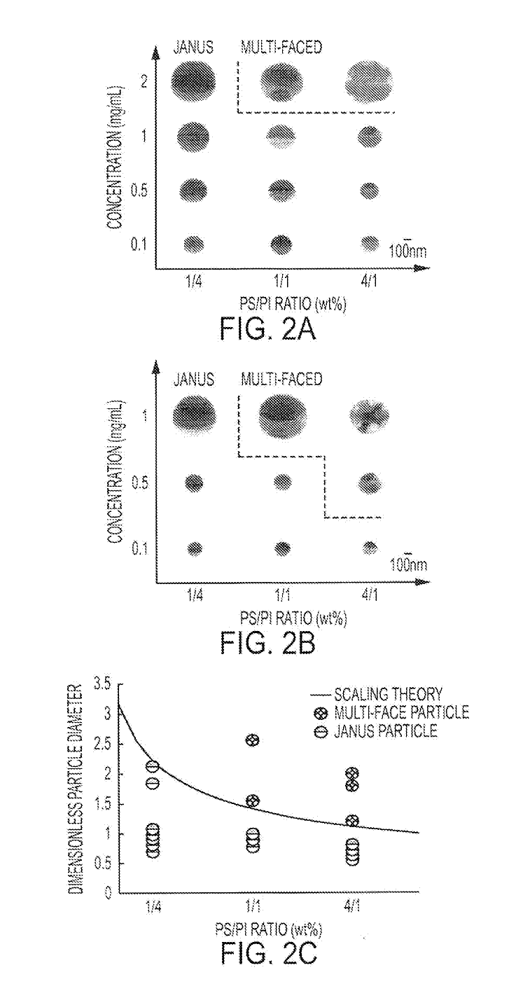

Janus nanocolloids of polystyrene (PS; Mw 16,500 g/mol) and polyisoprene (PI; Mw=11,000 g/mol) (χ_{PS-PI}=0.07) were formed (Physical Properties of Polymers Handbook, Springer, 2007, 349-355). Tetrahydrofuran (THF) and water were selected as the solvent and non-solvent, respectively. The process conditions employed, e.g., jet velocity ˜1 m/s and a 1 mm orifice, resulted in a Reynolds number −3500. Other mixing velocities, for example, in a range from 0.1 m/s to 30 m/s, resulting in other Reynolds numbers, for example, in a range from 300 to 100,000, can be used. For example, mixing velocities ranging from about 0.1 m/s, 0.3 m/s, 1 m/s, 3 m/s, or 10 m/s to about 0.3 m/s, 1 m/s, 3 m/s, 10 m/s, or 30 m/s can be used. For example, Reynolds numbers can range from about 300, 1000, 3000, 10,000, or 30,000 to about 1000, 3000, 10,000, 30,000, or 100,000. Symmetric Janus nanocolloids with a diameter (d) ˜200 nm were formed. To demonstrate the versatility of the process, PS-PI Janus nanocolloids of similar size but varying anisotropy were generated in a systematic manner (see, FIGS. 2A-2C). Simultaneous control over Janus particle size and spatial anisotropy was achieved by simply altering the homopolymer feed ratio and overall feed concentration. This was accomplished without the need for additional process modifications or surfactant interfacial stabilizers. The self-assembled nanocolloids instead acquired their stability from a colloidally-stabilizing surface charge of −33 mV that appears to have resulted from interactions between the surrounding aqueous media and the hydrophobic particle surface (Beattie, J. K. et al., The Surface of Neat Water is Basic, Faraday Discuss. 2009, 141, 31-39). Importantly, the absence of surfactants allows for fully Janus interior and exterior structures, unlike most surfactant-based particle formation processes.

An indispensable feature of the precipitation-induced self-assembly by the FNP (PISA-FNP) method is that key process parameters can be independently manipulated to understand their influence on particle size and morphology as well as gain insight into the mechanism of Janus nanocolloid formation. For instance, representative images of PS-PI Janus nanocolloids processed as a function of overall feed concentration and polymer ratio are shown in FIG. 2A, which provides TEM images of the particles with polystyrene (PS; Mw=16,500 g/mol) and polyisoprene (PI; Mw=11,000 g/mol). FIG. 2A illustrates that increasing the overall (total) feed concentration from 0.1 to 1.0 mg/mL systematically increases the size of the Janus nanocolloids from ˜200 nm to ˜600 nm in diameter. The particle anisotropy can furthermore be tuned independently at each feed concentration by altering the PS-PI polymer ratio from 20:80 to 80:20. The first ratio of components value is the percent of the total polymer mass that is the first polymer, and the second ratio of components value is the percent of the total mass that is the second polymer. Thus, sum of the first ratio and the second ratio is 100. Otherwise stated, the ratio is equal to the ratio of the polystyrene mass concentration to the polyisoprene mass concentration in the stream. As the overall feed concentration is increased to 2 mg/mL, the ability to form Janus nanocolloids depends on the feed ratio of PS to PI. At low PS/PI feed ratios, Janus colloids are observed. However, as the PS/PI feed ratio increases, multi-faced colloids are observed.

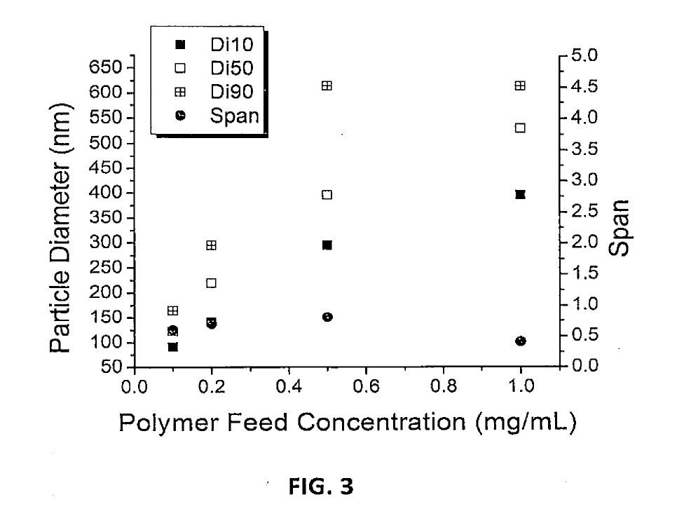

FIG. 3 shows size and polydispersity characteristics of nanoparticles formed for a 50:50 (that is, one-to-one) ratio of polystyrene mass concentration to polyisoprene mass concentration in the feed as a function of total polymer feed concentration. The left-hand axis indicates particle diameter. The Di10, Di50, and Di90 values for the group of particles formed at a given feed concentration are shown by the solid square, hollow square, and crossed square symbols, respectively. As discussed in the context of FIG. 2A, as the total polymer feed concentration increases, the size of the particles formed increases, as defined by each of the Di10, Di50, and Di90 (except that the Di90 value remained constant when increasing total polymer feed concentration from 0.5 to 1.0 mg/mL). The right-hand axis of FIG. 3 indicates the Span (Span=(Di90−Di10)/Di50). The Span increased (indicating a broadening particle size distribution) as the total polymer feed concentration increased from 0.1 to 0.5 mg/mL, but then decreased to its lowest value (indicating the narrowest particle size distribution) as the total polymer feed concentration was increased further to 1.0 mg/mL.

The phase diagram presented in FIG. 2C suggests a competition between the timescales of polymer de-mixing in confined environments and the vitrification time of PS, as set by the volumetric flow rate. According to this hypothesis, manipulating the timescale of either polymer phase separation or solvent exchange can shift the phase boundary between Janus and multi-faced internal structures in a controlled manner.

To investigate this effect, the FNP process was operated under identical conditions, but with an increase in the polymer molecular weight. FIG. 2B shows representative images of PS-PI Janus nanocolloids processed as a function of polymer ratio and overall feed concentration in which the PS and PI Mw were greater, 1,500 kg/mol and 1,000 kg/mol, respectively. At a feed concentration of 0.1 mg/mL, Janus nanocolloids were observed, illustrating that even high Mw polymers have sufficient mobility at dilute concentrations to self-organize into fully segregated polymer domains prior to kinetic trapping. As the overall feed concentration was increased, multi-faced nanocolloids formed, particularly at high PS/PI feed ratios.

Without being bound by theory, the structural features observed in the processed nanocolloids are consistent with the suggestion that internal particle formation proceeds via the phase separation of viscous fluids in a confined environment (see, FIG. 2C). The equilibrium Janus structure adopted by PS and PI at low feed concentrations suggested that the two polymers self-organized into two de-mixed hemispherical domains in order to minimize the total interfacial energy of the ternary phase (polymer-polymer-liquid) system. The Janus morphology, therefore, emerged because the two polymers possessed similar interfacial energies with the THF/water solution (γ_{PS-water}≈γ_{PI-water}) and a low interfacial energy between themselves (γ_{PS-PI}<γ_{PS-water} and γ_{PI-water}), while still forming a stable contact angle. When either the feed concentration or molecular weight of the PS and PI was significantly increased, the timescale over which the polymers phase separated during the assembly process was sufficiently increased above the vitrification time of PS to trap the internal colloidal structure in a non-equilibrium multi-faced state. Since the rate of phase separation decreases by ˜N², where N is the degree of polymerization, the high Mw polymers could generate multi-faced structures at lower polymer feed concentrations than their low Mw counterparts (Bates, F. S., Polymer-Polymer Phase Behavior, Science, 1991, 251, 898-905), The role of PS as a structural trapping agent, moreover, allowed for the enhanced capture of non-equilibrium structures in particles with a high PS content.

The morphology phase diagram for the particles (see, FIG. 2C) is consistent with a simple scaling theory based on surface nucleation (Cogswell, D. A. et al., Theory of Coherent Nucleation in Phase-Separating Nanoparticles, Nano Lett. 2013, 13, 3036-3041). The nearly uniform particle size R(c) for different PS:PI mixtures at the same feed-stream solvent concentration (c) suggests that phase separation occurs mainly after the flash precipitation of homogeneous particles. Spinodal decomposition would lead to random snake-like structures (Balluffi, R. W. et al., Kinetics of Materials, Wiley, 2005) or coherent stripes (Cogswell, D. A. et al., Coherency Strain and the Kinetics of Phase Separation in LiFePO₄Nanoparticles, ACS Nano 2012, 6, 2215-2225) that are not observed under these process conditions. A potential mechanism is heterogeneous nucleation by surface wetting (Cogswell, D. A. et al., Theory of Coherent Nucleation in Phase-Separating Nanoparticles, Nano Lett. 2013, 13, 3036-3041). While binary solids tend to favor complete coverage of each facet by a single phase (Cogswell, D. A. et al., Theory of Coherent Nucleation in Phase-Separating Nanoparticles, Nano Lett. 2013, 13, 3036-3041), the viscous polymer mixture exhibits partial wetting, so the observed nonequilibrium structures could result from confined capillary instability of the nucleated surface layer, arrested by PS vitrification. Relaxation to the Janus structure occurs if the PS diffusion distance \sqrt{Dτ} during the vitrification time τ exceeds the surface layer coalescence distance, scaling as:

$$R ( c ) {( 1 + \frac{PI}{PS} )}^{- 1 \text{/} 2} \qquad ( 1 )$$

This dimensionless criterion

$$\tilde{R} = \frac{R}{\sqrt{D τ}} < \sqrt{1 + \frac{PI}{PS}} \qquad ( 2 )$$

successfully predicts (solid line is scaling prediction) the formation of Janus versus patchy particles as shown in FIG. 2C, by collapsing the experimental data from FIGS. 2A and 2B.
Feed concentrations between 0.01 mg/mL and 50 mg/mL can be used for Janus particle formation. For example, feed concentrations from about 0.01, 0.02, 0.05, 0.1, 0.2, 0.5, 1, 2, 5, 10, or 20 mg/mL to about 0.02, 0.05, 0.1, 0.2, 0.5, 1, 2, 5, 10, 20, or 50 mg/mL can be used. The concentration at the upper end is determined by the time required for initial polymer aggregation. Too high of a concentration does not allow uniform particle formation. The lower concentration is determined by the cost of separations of such dilute final products. Concentrations between 0.1 mg/mL and 10 mg/mL are preferred. Polymer ratios between 0.5:99.5 and 99.5:0.5 are possible, depending on the desired application. For example, polymer ratios from about 0.5:99.5, 1:99, 2:98, 5:95, 10:90, 20:80, 30:70, 40:60, 50:50, 60:40, 70:30, 80:20, 90:10, 95:5, 98:2, or 99:1 to about 1:99, 2:98, 5:95, 10:90, 20:80, 30:70, 40:60, 50:50, 60:40, 70:30, 80:20, 90:10, 95:5, 98:2, 99:1, or 99.5:0.5 are possible. The lower and higher limits are determined by the requirement of polymer immiscibility between the two polymers to enable the final Janus particle to have two or more distinct phases. For particles with more than two components the compositions are specified as weight percent ratios, where the sum of the compositions of the components sums to 100.

### Example 3

### Janus Particles with Varied Polymer End-Group Functionality and Alternative Polymers

While the surface structure of nanocolloids strongly influences functionality, the material composition of surface domains determines the types of interactions the colloids exhibit with external environments. The PISA-FNP methodology has been extended to two other classes of systems: i) PS-PI Janus nanocolloids with varying polymer end-group functionality; and ii) Janus nanocolloids with new polymer components.

(i) PS-PI Janus nanocolloids were prepared with polymer surfaces containing varying amounts of hydrogen or hydroxyl moieties. This was achieved by using homopolymers with different end-group functionalities in the feed stream, rather than chemically altering the particles post-fabrication. The particles prepared included the following Janus particles: particles prepared with polystyrene and hydroxy-terminated polybutadiene; particles prepared with hydroxy-terminated polystyrene and hydroxy-terminated polybutadiene; and particles prepared with hydroxy-terminated polystyrene and polybutadiene. PS-PI Janus nanocolloids with carboxyl functionalities were also demonstrated. The surface functionality of the Janus colloids can thus be systematically tuned accordingly and allows for further chemical modification of the particles as needed for specific applications.

(ii) The full domain composition was modified to form Janus particles from polystyrene (PS)-poly(lactic acid) (PLA) and polybutadiene (PB)-PLA polymer pairings. The process is therefore capable of producing Janus nanocolloids from varied polymer combinations, including those consisting of biodegradable materials, despite dissimilarity between the properties of paired polymer components.

Janus particles can be formed from polymers selected from other polymers in addition to polystyrene (PS), polyisoprene (PI), polybutadiene (PB), poly(lactic acid) (PLA), poly(vinylpyridine) (PVP), polyvinylcyclohexane poly(methacrylic acid), poly(methyl methacrylate), polycaprolactone, polyamide (e.g., nylon 6,6), polysulfone, epoxy, epoxy resin, silicone polymer, silicone rubber, and polyimide. Polymers used to form Janus particles may be synthetic polymers or natural products. In addition, co-polymers of these polymers may be used, as long as the copolymers phase separate from each other to form multidomain structures. Furthermore, mixtures of polymers may be employed where one or more polymers are miscible into a “first polymer phase”, but the additional polymer(s) that form a “second polymer phase” are immiscible with the first polymer phase. In addition, for multicomponent particles, multiple polymers and mixtures may be used, as long as the polymer phases separate into multiple phases. Many pairs of polymers that are immiscible can be used to form Janus particles. As discussed herein, one or more block copolymers formed from copolymers can be used to form Janus particles. Some additional polymers that can be used to from Janus particles are hydrophobic polymers and hydrophobic polymers formed from moieties such as acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylatcs, aminoalkylmethacrylates, and aminoalkyl(meth)acrylamides; styrenes; cellulose acetate phthalate, cellulose acetate succinate, hydroxypropylmethylcellulose phthalate, and the polymers poly(D,L lactide), poly(D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Illum, L., Davids, S. S. (eds.) Polymers in Controlled Drug Delivery, Wright, Bristol, 1987; Arshady, J. Controlled Release (1991) 17, 1-22; Pitt, Int. J. Phar. (1990) 59, 173-196; Holland, et al., J. Controlled Release (1986) 4, 155-180); hydrophobic peptide-based polymers and copolymers based on poly(L-amino acids) (Lavasanifar, A., et al., Advanced Drug Delivery Reviews (2002) 54, 169-190), poly(ethylene-vinyl acetate) (“EVA”) copolymers, silicone rubber, polyethylene, polypropylene, polydienes (e.g., hydrogenated forms of polybutadiene and polyisoprene), maleic anhydride copolymers of vinyl-methylether and other vinyl ethers, polyurethane, poly(ester urethanes), poly(ether urethanes), poly(ester-urea). For example, preferred polymeric hydrophobes include poly(ethylenevinyl acetate), poly (D,L-lactic acid) oligomers and polymers, poly (L-lactic acid) oligomers and polymers, poly(glycolic acid), copolymers of lactic acid and glycolic acid, poly (valerolactone), polyanhydride, and copolymers of poly(caprolactone) or poly(lactic acid). For non-biologically related applications preferred polymers include polystyrene, polyacrylate, butadiene, polysiloxane, polyamide, and polyester.

### Example 4

### Cerebus Particles

One of the advantages of the PISA-FNP system is that more than two homopolymers can be simultaneously fed into the system, opening the possibility of generating complex nanocolloids not previously fabricated by a facile bottom-up self-assembly approach. Feeding an equal ratio of three immiscible polymers PS (Mw=16,500 g/mol), PB (Mw=18,000 g/mol), and poly(vinyl cyclohexane) (PVCH) (Mw=25,000 g/mol) dissolved in THF and co-precipitated with an aqueous non-solvent led to the formation of patchy, tri-lobal nanocolloids (χ_{PS-PI}=0.07, χ_{PS-PVCH}=0.32) (Beckingham, B. et al., Regular Mixing Thermodynamics of Hydrogenated Styrene-Isoprene Block-Random Copolymers, Macromolecules 2013, 3084-3091). FIG. 4 shows a TEM image of a tri-lobal Cerberus particle possessing three phase-separated polymer surface domains composed of polystyrene, polybutadiene (dark middle region), and polyvinylcyclohexane (a digital rendering of the particle is also shown).

### Example 5

### Metal-Polymer Nanoparticles

A process according to the invention generates metal-polymer nanoparticles through a one-step, self-assembly approach. That is, the self-assembly process is simplified by synergistically preparing hybrid metal nanoparticle deposited onto spherical polymer assemblies, namely, metal on polystyrene-b-poly(4-vinylpyridine) (metal-PS-b-PVP) through the one-step continuous route of Flash NanoPrecipitation (FNP). Polystyrene-block-poly(vinylpyridine) (PS-b-PVP) in THF, aqueous gold salts, and reducing agent solutions are employed as the polymer stream, non-solvent stream, and collection solution, respectively. Uniform Au nanoparticles deposited on polymer nanospheres are then obtained through a one-step FNP process. Stabilizers, such as sodium dodecyl sulfate (SDS), may be added to the process, for example, to the collection solution, to further control stability.

A confined impinging jet mixer composed of two separate streams was used. A syringe containing 1 mL of 3 mg/mL PS₇₉₃_{^{k}}-b-PVP₃ₛₖ (PDI=1.08, purchased from Polymer Source, Inc.) in THF was placed at the inlet of Stream 1, and a syringe containing 1 mL of 0.45 mg/mL chloroauric acid (HAuCl₄) in H₂O was placed at the inlet of Stream 2. Subsequently, fluid was expelled manually from both syringes at the same rate (˜1 mL per second), causing the two streams to merge into a mixing stream. The mixing stream was diluted into a 10 mL water reservoir containing 1 mg of the reducing agent, sodium borohydride (NaBH₄), and 10 mg of the stabilizer, sodium dodecyl sulfate (SDS). The water reservoir quenched the precipitated nanoparticles, and a stable colloid solution was formed. In a separate experiment, Pt@PS-b-PVP was prepared by replacing Stream 2 with 1 mL of 3 mg/mL hexachloroplatinic acid (H₂PtCl₆) in water (H₂O).

Scanning electron microscopy (SEM) shows that the obtained Au@PS-b-PVP (PI) composites have regular spherical morphology with a uniform diameter of ˜80 nm. The dynamic light scattering (DLS) data and a photograph of colloid solution show that the obtained composites are well dispersed in water with hydrodynamic diameter ˜100 nm. A transmission electron microscopic (TEM) image confirmed that the Au@PS-b-PVP nanoparticles are monodisperse and uniformly spherical, with a mean diameter of ˜80 nm. Furthermore, the characteristics of the individual nanoparticles were visualized by TEM. The Au@PS-b-PVP nanoparticle consists of a predominantly PS core, a thin PVP shell and Au nanocrystals (NCs) uniformly embedded in the PVP layer. The diameter of Au NCs was about 3 nm and the 0.23 urn lattice spacing corresponding to the (111) plane of face-centered cubic (fcc) structure of gold nanoparticles was visually confirmed. All the Au NCs were located near the surface of the polymeric support, and aggregation of the Au NCs on the surface was minimal.

In order to confirm that the PVP segment of the PS-b-PVP block copolymer contributes to the formation of the composite, a control experiment was carried out by using PS homopolymer (Mw=376 kg/mol) dissolved in THF as Stream 1. A large amount of free Au NCs are formed while no Au NCs are observed on the surface of the polymer nanoparticles. The above observation and comparison indicate that the AuCl₄⁻ ions in the system strongly localize to and disperse well within the PVP corona of the polymer particles due to the complexation of the AuCl₄⁻ ions with the pyridine units of the PVP blocks (Spatz, J. P. et al., Langmuir 2000, 16, 407; Spatz, J. P. et al., Adv. Mater. 2002, 14, 1827; Suntivich, P. et al., Langmuir 2011, 27, 10730; Leong, W. L. et al., Adv. Mater. 2008, 20, 2325).

Without being bound by theory, the process of nanoparticle formation can be envisioned as follows. PS-b-PVP block polymers self-assemble into nanoparticles with PS as the predominant core and PVP as the corona when the two input streams mix in the confined chamber. Subsequently, AuCl₄⁻ ions are attracted into the PVP network. The ion-block copolymer complex disperses into a water reservoir containing NaBH₄ that reduces the entrapped AuCl₄⁻ ions into Au seeds. The growth of gold within the PVP layer then takes place and the complex is quenched to form stable Au@PS-b-PVP composites. The presence of SDS in the water reservoir inhibits particle coalescence while the absence of SDS results in nanoparticle aggregation within minutes. The reported methodology has also been demonstrated in the successful fabrication of Pt@PS-b-PVP nanoparticles when using H₂PtCl₆ in H₂O as Stream 2: ˜2 nm Pt nanocrystals uniformly deposit on the surface of the polymer nanoparticles.

The effect of processing parameters on the preparation of the composites was studied by varying polymer and gold salt concentrations, as shown in Table 2 below.

TABLE 2

| | Stream 1 | Stream 2 |
|---|---|---|
| | PS-b-PVP/THF | HAuCl₄/H₂O |
| | (mg/mL) | (mg/mL) |
| P1 | 3 | 0.45 |
| P2 | 3 | 0.15 |
| P3 | 3 | 0.9 |
| P4 | 6 | 0.45 |
| P5 | 1 | 0.45 |
| P6 | 1 | 0.15 |

The composites P2 and P3 were prepared by feeding 0.15 mg/mL and 0.9 mg/mL HAuCl₄ concentrations, respectively, in Stream 2. The two composites along with P1, created from 0.45 mg/mL HAuCl₄ feed concentration, have nearly the same composite diameter and size distribution independent of the Au salt concentration in the feed. This was also confirmed by DLS measurements. Increasing the HAuCl₄ feed concentration to 0.9 mg/mL produced a denser distribution of Au nanocrystals (NCs) on the polymer surface and slightly increased the Au NC sizes to ˜5 nm. Lowering the HAuCl₄ feed concentration to 0.15 mg/mL resulted in a sparser distribution of Au nanocrystals. The UV-Vis absorbance of the composites was measured and investigated for the surface plasmon resonance (SPR) peak associated with the presence of Au nanoparticles. The spectrum of P2 did not show an obvious plasmon band, indicating a discrete distribution of Au NCs. As the HAuCl₄ concentration in the feed was increased, a sharper and more intense plasmon absorption band with a slight red shift is observed, which was thought to result from the larger Au nanocrystals as well as the reduced Au nanoparticle interspacing. (Jana, N. R. et al., Langmuir 2001, 17, 6782.; Hussain, I. et al., J. Am. Chem. Soc. 2005, 127, 16398; Rao, T. L. et al., Soft Matter 2012, 8, 2963.)

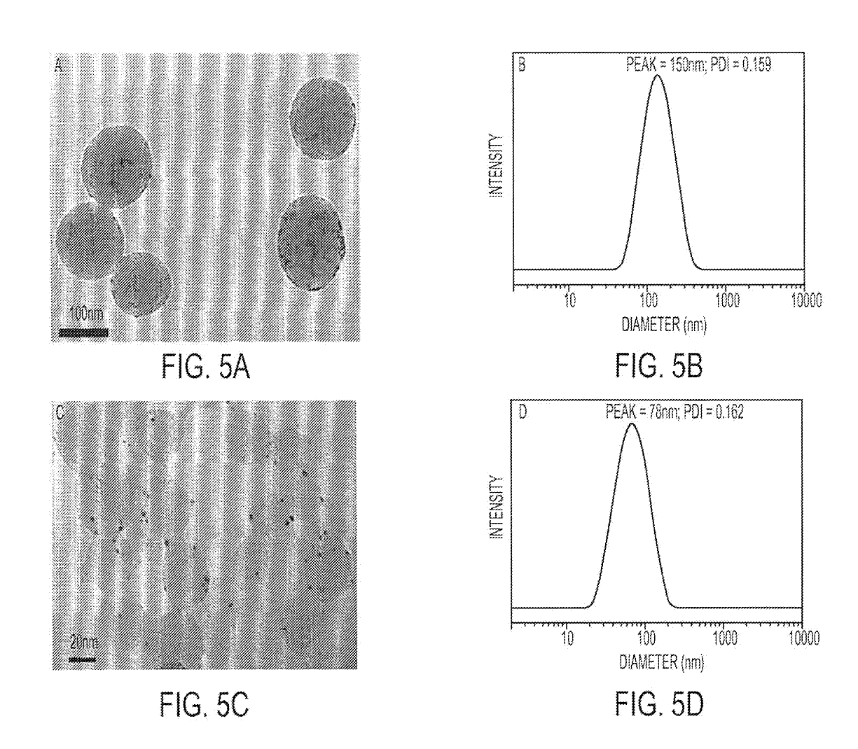

The overall size of the composites, on the other hand, was controlled by varying the PS-b-PVP concentration in the THF stream. FIGS. 5A and 5B show a TEM image and DLS data, respectively, of P4 particles prepared with 6 mg/mL polymer concentration. As the polymer concentration increased, the nanoparticles also increased in size to ˜120 nm while remaining monodisperse. Similarly, lowering the feed polymer concentration to 1 mg/mL reduced the size of P5 to ˜30 nm. The large Au clusters in PS are formed on the polymer surface due to the higher local concentration of gold salts per polymer nanoparticle. Decreasing the feed polymer and gold salt concentrations to 1 mg/mL and 0.15 mg/mL, respectively, led to ˜30 nm nanoparticles (P6) with a distribution of well-dispersed Au NCs on the surface, as shown by the TEM image and DLS data of FIGS. 5C and 5D, respectively. Some polymer composite aggregates form in P5 and P6 at a polymer concentration of 0.15 mg/mL regardless of gold salt concentration, which led to a large hydrodynamic diameter of ˜80 nm.

### Example 6

### Catalysis with Metal-Polymer Nanoparticles

The gold-catalyzed reduction of 4-nitrophenol by NaBH₄ to 4-aminophenol was used as a model reaction to evaluate the catalytic capability of the synthesized Au@PS-b-PVP hybrid nanoparticles (P1). 1 mL of 0.1 mM 4-nitrophenol was mixed with a freshly prepared aqueous solution of NaBH₄ (2 mL, 0.1 M). Au@PS-b-PVP (P1) (250 μg) was then added. UV/Vis absorption spectra were recorded to monitor the change in the reaction mixture after the removal of nanoparticles.

The reduction reaction did not proceed without the presence of Au@PS-b-PVP catalyst, as evidenced by a constant absorption peak at 400 nm. However, when Au@PS-b-PVP catalyst was introduced into the solution, the absorption at 400 nm quickly decreased while the absorption at 295 nm increased. The reduction of 4-nitrophenol into 4-aminophenol was completed in ˜1 min. The complete conversion of 4-nitrophenol could also be visually appreciated by the color change of the solution from yellow to clear.

Stability against coalescence is an important issue for nanocrystal-based catalysts. (Comotti, M. et al., Angew. Chem. 2004, 116, 5936; Angew. Chem. Int. Ed. 2006, 45, 8224; Valdés-Solís, T. et al., J. Catal. 2007, 251, 239.) The stability of Au@PS-b-PVP was investigated by repeating the reduction reaction with the same catalyst for five cycles. After each reaction, the catalyst was recycled by centrifugation, followed by washing with distilled water and drying in vacuum overnight at room temperature. The catalyst showed high activity after five successive cycles of reactions, with conversion close to 100% within ˜1 min of reaction time. The well-dispersed Au NCs were still visualized by TEM on the surface of the polymer support after five reaction cycles. Thus, the composites with gold nanoparticles embedded in the corona were effective at preventing the coalescence of catalyst nanoparticles, making the catalyst reusable after multiple cycles of reactions. Combining the advantages of facile synthesis and large scale production, FNP is an attractive platform for the production of stable and recyclable nanocatalysts.

In summary, hybrid Au (or Pt) on PS-b-PVP assemblies were produced through Flash NanoPrecipitation. Control over the nanoparticle size, gold cluster size, and overall optical response was achieved by changing the process parameters. The hybrid nanoparticles exhibited high catalytic performance and good reusability for the reduction of 4-nitrophenol. The PS-b-PVP used can be replaced by conducting, biodegradable, or binary blends of polymer to facilitate the simultaneous entrapment of various functional (e.g., magnetic or fluorescent) cores during the precipitation process.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### REFERENCES

Sharma G. et al., Macromol. Rapid Commun. 2004, 25, 547.
Mei, Y. et al., Langmuir 2005, 21, 12229.
Budijono, S. et al., Nanoparticles for photodynamic therapy: Google Patents; 2009 (PCT/US2008/012485 and US20110022129).
Burke, P. A. et al., Preparation of lipid nanoparticles: Google Patents; 2011 (PCTTUS2011/031540 and US20130037977).
Kumar, V. et al., Preparation of lipid nanoparticles: Google Patents; 2013 (EP2558074).
Mayer, L. D. et al., Particulate constructs for release of active agents: Google Patents; 2006 (PCT/US2005/025549).
Mayer, L. D. et al., Particulate constructs for release of active agents: Google Patents; 2007 (EP1786443).
Prud'homme, R. K. et al., Lung targeting dual drug delivery system: Google Patents; 2011 (US20110268803).
York, A. W. et al., Bioactive amphiphilic polymer stabilized nanoparticles with enhanced stability and activity: Google Patents; 2013 (PCT/US2012/050040).
Gindy, M. et al., Preparation of Lipid Nanoparticles, US Patent Publication, US20130037977 At, PCT/US2011/031540, publication date Feb. 14, 2013.
Figureroa, C. et al., A high-loading nanoparticle-based formulation for water-insoluble steroids, Patent Publication, WO2013063279 A1, PCT/US2012/061945, publication date May 2, 2013.
Mayer, L. D. et al., Particulate constructs for release of active agents, Patent Publication US20130336915 A1, publication date Dec. 19, 2013.
Johnson, B. K. et al., Process and Apparatuses for Preparing Nanoparticle Compositions with Amphiphilic Copolymers and Their Use, US Patent Application Pub., US 2012/0171254 A1, Jul. 5, 2012.

1. A method of forming a multi-faced polymer nanoparticle, comprising
dissolving a first polymer at a first concentration and a second polymer at a second concentration in a solvent to form a polymer solution,
selecting a nonsolvent,
selecting a mean nanoparticle diameter,
selecting the first concentration and second concentration to achieve the selected moan nanoparticle diameter, and
continuously mixing the polymer solution with the nonsolvent to flash precipitate the multi-faced polymer nanoparticle in a mixture of the solvent and the nonsolvent,
wherein the first polymer is different from the second polymer and
wherein the multi-faced polymer nanoparticle comprises
a first region, comprising the first polymer at a greater weight fraction than the second polymer, and
a second region, comprising the second polymer at a greater weight fraction than the first polymer,
the first region being in contact with the second region.

2. The method of claim 1, wherein neither the polymer solution nor the nonsolvent comprise a stabilizer.

3. The method of claim 1, wherein the mixing of the polymer solution with the nonsolvent further comprises mixing with a collection solution.

4. The method of claim 3, wherein the collection solution comprises a stabilizer.

5. The method of claim 4, wherein the stabilizer is an amphiphilic surfactant molecule.

6. The method of claim 4, wherein the stabilizer is selected from the group consisting of sulfonated alkyl surfactants, sodium dodecyl sulfate, ethoxylated sulfonate surfactants, cationic surfactants, amine oxide surfactants, zwitterionic surfactants, amphoteric surfactants, ethylene oxide surfactants based on alkyl ethers, ethylene oxide surfactants based on nonylphenols, surfactants based on sorbitan oleates, glucose-based surfactants, polymeric surfactants, polyethylene oxide-co-polybutylene oxide surfactants, polyvinyl caprolactam based stabilizers, polycaprolactone based stabilizers, polyvinyl alcohol based stabilizers, polyethylene oxide based stabilizers, natural products polymeric stabilizers based on substituted cellulose, hydroxypropyl cellulose, natural products polymeric stabilizers based on hydrophobically modified starches, lipids, lecithin, and combinations.

7. The method of claim 1, wherein the mean particle diameter is in a range of 30 nm to 2000 nm.

8. The method of claim 1, wherein the mean particle diameter is in a range of 50 nm to 800 nm.

9. The method of claim 1, wherein at least 90% of the nanoparticles formed have a diameter less than 800 nm and at most 10% of the nanoparticles formed have a diameter less than 50 nm.

10. The method of claim 1,
wherein the multi-faced polymer nanoparticle has a total volume and
wherein the first region and the second region together comprise at least 90% of the total volume.

11. The method of claim 1,
wherein the first polymer is selected from the group consisting of polystyrene (PS), polyisoprene (PI), polybutadiene (PB), poly(lactic acid) (PLA), poly(vinylpyridine) (PVP), polyvinylcyclohexane, poly(methyl methacrylate), polycaprolactone, polyamide, polysulfone, epoxy resin, silicone polymer, and polyimide,
wherein the second polymer is selected from the group consisting of polystyrene (PS), polyisoprene (PI), polybutadiene (PB), poly(lactic acid) (PLA), poly(vinylpyridine) (PVP), polyvinylcyclohexane (PVCH), poly(methyl methacrylate), polycaprolactone, polyamide, polysulfone, epoxy resin, silicone polymer, and polyimide,
wherein the first concentration is in the range from 0.01 to 30 mg/mL,
wherein the second concentration is in the range from 0.01 to 30 mg/mL,
wherein the solvent is selected from the group consisting of tetrahydrofuran (THF), methyl acetate, ethyl acetate, acetone, methyl ethyl ketone (MEK), dioxane, dimethylformamide (DMF), acetonitrile, methyl pyrrolidone, and dimethyl sulfoxide (DMSO) and combinations, and
wherein the nonsolvent is selected from the group consisting of water, methanol, ethanol, acetic acid and combinations.

12. The method of claim 11,
wherein the first polymer is polystyrene (PS),
wherein the second polymer is polyisoprene (PI),
wherein the solvent is tetrahydrofuran (THF), and
wherein the nonsolvent is water.

13. The method of claim 1,
wherein the first polymer is poly(methacrylic acid),
wherein the solvent is water, and
wherein the nonsolvent is acetone.

14. The method of claim 1, further comprising
dissolving an amphiphilic block copolymer in the solvent,
wherein the amphiphilic block polymer comprises a hydrophobic homopolymer covalently bonded to a hydrophilic homopolymer, the hydrophobic homopolymer having the same chemical structure as the first polymer.

15. The method of claim 14, further comprising
dissolving a second amphiphilic block copolymer in the solvent,
wherein the second amphiphilic block polymer comprises a second hydrophobic homopolymer covalently bonded to a hydrophilic homopolymer, the second hydrophobic homopolymer having the same chemical structure as the second polymer.

16. The method of claim 1, further comprising separating the multi-faced polymer nanoparticle from the mixture.

17. The method of claim 16, wherein the multi-faced polymer nanoparticle is separated from the mixture by a procedure selected from the group consisting of centrifugation, ultrafiltration, spray drying, and combinations.

18. The method of claim 1, further comprising infusing the multi-faced nanoparticle with a medical agent.

19. The method of claim 18, wherein the medical agent is selected from the group consisting of a pharmaceutical, an imaging agent, a contrast imaging agent, and a radioactive tracer.

20. The method of claim 1, further comprising infusing the multi-faced nanoparticle with a pesticide or an herbicide.

21. The method of claim 1,
wherein the first polymer is a homopolymer or a near homopolymer,
wherein the near homopolymer comprises a first comonomer and a second comonomer,
wherein the first comonomer is at least 95 wt % of the near homopolymer, and
wherein the second comonomer is at most 5 wt % of the near homopolymer.

22. The method of claim 21, wherein the mixing of the polymer solution with the nonsolvent further comprises mixing with a collection solution comprising an anionic surfactant.

23. A plurality of multi-faced polymer nanoparticles,
wherein each multi-faced polymer nanoparticle comprises
a first polymer,
a second polymer,
a first region, comprising the first polymer at a greater weight fraction than the second polymer, and
a second region, comprising the second polymer at a greater weight fraction than the first polymer,
wherein the first region is in contact with the second region and
wherein at least 80% of the particles have a diameter in the range of from 50 nm to 800 nm.

24. The plurality of multi-faced polymer nanoparticles, wherein the first polymer is a biocompatible polymer.

25. A method of using the plurality of multi-faced polymer nanoparticles of claim 23 to strengthen adhesion between a first polymer structure and a second polymer structure at an interface between the first polymer structure and the second polymer structure.

26. A method of using the plurality of multi-faced polymer nanoparticles of claim 23 as an emulsion stabilizer.

27. A method of using the plurality of multi-faced polymer nanoparticles of claim 23 as a foam stabilizer.

28. A method of using the plurality of multi-faced polymer nanoparticles of claim 23 as a foam stabilizer.

29. A method of using the plurality of multi-faced polymer nanoparticles of claim 23 as a solid-liquid interfacial tension modifier.

30. A three-faced polymer nanoparticle, comprising
a first polymer,
a second polymer,
a third polymer,
a first region, comprising the first polymer at a greater molar fraction than a molar fraction of the second polymer and third polymer,
a second region, comprising the second polymer at a greater molar fraction than a molar fraction of the first polymer and third polymer, and
a third region, comprising the third polymer at a greater molar fraction than a molar fraction of the first polymer and second polymer,
wherein the first region is in contact with the second region,
wherein the second region is in contact with the third region, and
wherein each of the first polymer, second polymer, and third polymer are different from each other.

31. The three-faced polymer nanoparticle of claim 30,
wherein the three-faced polymer nanoparticle has a total volume and
wherein the first region, second region, and third region together comprise at least 90% of the total volume.

32. The three-faced polymer nanoparticle of claim 30,
wherein the first polymer is polyvinylcyclohexane (PVCH),
wherein the second polymer is polybutadiene (PB), and
wherein the third polymer is polystyrene (PS).

33. A method for forming a metal-polymer composite nanoparticle, comprising
dissolving a polymer in a first solvent at a first concentration to form a polymer solution,
dissolving a metal salt in a second solvent at a second concentration to form a metal salt solution, and
mixing the polymer solution with the metal salt solution to form a metal-polymer composite nanoparticle having a surface,
wherein metal is concentrated at the surface and
wherein the second solvent is a nonsolvent for the polymer.

34. The method of claim 33, wherein the polymer is a block copolymer.

35. The method of claim 33, wherein the polymer is polystyrene-block-poly(vinylpyridine) (PS-b-PVP).

36. The method of claim 33, wherein the metal is selected from the group consisting of gold (Au), platinum (Pt), silver (Ag), palladium (Pd), copper (Cu), cobalt (Co), iron (Fe), and combinations.

37. The method of claim 33,
wherein the mixing of the polymer solution with the metal salt solution further comprises mixing with a collection solution.

38. The method of claim 37,
wherein the collection solution comprises a reducing agent.

39. The method of claim 38, wherein the reducing agent is selected from the group consisting of lithium aluminum hydride (LiAlH₄), compounds containing the Sn²⁺ ion, tin(II)chloride (SnCl₂), compounds containing the Fe²⁺ ion, iron (II) sulfate (FeSO₄), oxalic acid, formic acid, ascorbic acid, sulfite compounds, phosphites, hydrophosphites, phosphorous acid, dithiothreitol (DTT), tris(2-carboxyethyl)phosphine HCl (TCEP), carbon, and combinations.

40. The method of claim 38, wherein the reducing agent is sodium borohydride (NaBH₄)

41. The method of claim 37, wherein the collection solution comprises a stabilizer.

42. The method of claim 41,
wherein the stabilizer is sodium dodecyl sulfate (SDS).

43. A metal-polymer composite nanoparticle, comprising
a core and
a shell that surrounds the core,
wherein the core comprises a polymer and
wherein the shell comprises the polymer and a metal.

44. The metal-polymer composite nanoparticle of claim 43, wherein the polymer is a block copolymer.

45. A method of using the metal-polymer composite nanoparticle of claim 43 to catalyze a chemical reaction.

46. A method of using the metal-polymer composite nanoparticle of claim 43 to catalyze a chemical reaction between two immiscible phase liquids.

47. A multi-faced polymer nanoparticle comprising,
a first polymer,
a second polymer,
a first region, comprising the first polymer at a greater molar fraction than the second polymer, and
a second region, comprising the second polymer at a greater molar fraction than the first polymer,
wherein the first region is in contact with the second region,
wherein the first polymer is a homopolymer or a near homopolymer,
wherein the near homopolymer comprises a first comonomer and a second comonomer,
wherein the first comonomer is at least 95 wt % of the near homopolymer, and
wherein the second comonomer is at most 5 wt % of the near homopolymer.
